(19) United States
(12) Patent Application Publication
(10) Pub. No.: US 2023/0348595 A1
(43) Pub. Date: Nov. 2, 2023

(54) SOLUBLE TCRS AND FUSIONS TO ANTI-CD3 RECOGNIZING KRAS G12D FOR THE TREATMENT OF CANCER

(71) Applicant: IMMUNOCORE LIMITED, Abingdon, Oxfordshire (GB)

(72) Inventors: Chandramouli CHILLAKURI, Abingdon, Oxfordshire (GB); Andrew POOLE, Abingdon, Oxfordshire (GB); Sarah BAILEY, Abingdon, Oxfordshire (GB)

(21) Appl. No.: 17/998,032

(22) PCT Filed: May 4, 2021
(86) PCT No.: PCT/EP2021/061731

§ 371 Date: Nov. 5, 2022

(30) Foreign Application Priority Data
May 5, 2020 (GB) 2006629.6

(51) Int. Cl. C07K 16/28; A61P 35/00

(52) U.S. Cl. CPC C07K 16/2809; A61P 35/00; C07K 16/2833; C07K 2317/565; C07K 2317/567; C07K 2319/32

(57) ABSTRACT

The present invention relates to specific binding molecules which bind to an HLA-restricted peptide derived from mutant KRAS. Said specific binding molecules may comprise CDR sequences embedded within a framework sequence. The CDRs and framework sequences may correspond to a T cell receptor (TCR) variable domain and may further comprise non-natural mutations relative to a native TCR variable domain. The specific binding molecules of the invention are particularly suitable for use as novel immunotherapeutic reagents for the treatment of cancer.

The present invention relates to specific binding molecules which bind to the HLA-restricted peptide VVVGADGVGK (SEQ ID NO: 1) derived from mutant KRAS. Said specific binding molecules may comprise CDR sequences embedded within a framework sequence. The CDRs and framework sequences may correspond to a T cell receptor (TCR) variable domain and may further comprise non-natural mutations relative to a native TCR variable domain. The specific binding molecules of the invention are particularly suitable for use as novel immunotherapeutic reagents for the treatment of cancer.

### BACKGROUND TO THE INVENTION

Kirsten rat sarcoma viral oncogene homolog (KRAS) is a ubiquitously expressed small GTPase that drives cell signalling, survival and proliferation of downstream of growth factor receptors (Uniprot no: P01116). Oncogenic, somatic gain of function mutations in KRAS are well described in the literature and have been reported to be present in approximately 20% of all human cancers, including for example, pancreatic, colorectal, lung, endometrial, ovarian, and prostate cancers (Cox et al., Nat Rev Drug Discov. 2014 Nov;13(11):828-51). A single amino acid substitution can be responsible for giving rise to the mutated KRAS. In particular, a mutation at position G12 of KRAS is reported to comprise 83% of all mutations (Hobbs et al., Cancer Cell. 2016 Mar 14;29(3):251-253). Both G12D and G12V mutations are common in pancreatic and colon cancer. A number of small molecule drugs have been developed to target G12 mutated KRAS, but as yet, none has been approved for therapeutic use. Therefore there is a need for more effective drugs to target mutated KRAS, and also for alternatives to small molecule drugs.

T cell receptors (TCRs) recognize short peptide antigens that are displayed on the surface of antigen presenting cells in complex with Major Histocompatibility Complex (MHC) molecules (in humans, MHC molecules are also known as Human Leukocyte Antigens, or HLA) (Davis et al., Annu Rev Immunol. 1998;16:523-44). TCRs that target the HLA-A*11 restricted peptide VVVGADGVGK (SEQ ID No.1), derived from G12D mutant KRAS, are known in the art (Wang et al., Cancer Immunol Res. 2016 Mar; 4(3): 204-214). The development of TCR-based therapeutic reagents to target VWGADGVGK-HLA-A*11 complex is challenging, since the TCR must be able to adequately discriminate between the mutated (tumour) peptide and the non-mutated wildtype peptide, which differs by only one amino acid. Cross recognition of the wild type peptide may lead to undesirable targeting of normal healthy tissues.

### DESCRIPTION OF THE INVENTION

In a first aspect, the present invention provides a specific binding molecule having the property of binding to VWGADGVGK (SEQ ID NO: 1) in complex with HLA-A11 and comprising a TCR alpha chain variable domain and/or a TCR beta chain variable domain, each of which comprises FR1-CDR1-FR2-CDR2-FR3-CDR3-FR4, where FR is a framework region and CDR is a complementarity determining region, wherein
(a) the alpha chain CDRs have the following sequences:
   CDR1 — TRDTTYY (SEQ ID No: 32),
   CDR2 — RNSFDEQNE (SEQ ID No: 33),
   CDR3 — CALSGPSGAGSYQLTF (SEQ ID No: 34),
   optionally with one or more mutations therein, and/or
(b) the beta chain CDRs have the following sequences:
   CDR1 — MNHEY (SEQ ID No: 35),
   CDR2 — SVGEGT (SEQ ID No: 36),
   CDR3 — CASSYGPGQHNSPLHF (SEQ ID No: 37),
   optionally with one or more mutations therein.

In the specific binding molecule of the first aspect, the alpha chain variable domain framework regions may comprise the following framework sequences:
FR1 — amino acids 1-26 of SEQ ID NO: 2,
FR2 — amino acids 34-50 of SEQ ID NO: 2,
FR3 — amino acids 60-91 of SEQ ID NO: 2,
FR4 — amino acids 108-117 of SEQ ID NO: 2,
or respective sequences having at least 90, 91, 92, 93, 94, 95, 96, 97, 98 or 99% identity to said sequences, and/or the beta chain variable domain framework regions may comprise the following sequences:
FR1 — amino acids 1-26 of SEQ ID NO: 3,
FR2 — amino acids 32-48 of SEQ ID NO: 3,
FR3 — amino acids 55-90 of SEQ ID NO: 3,
FR4 — amino acids 106-115 of SEQ ID NO: 3,
or respective sequences having at least 90, 91, 92, 93, 94, 95, 96, 97, 98 or 99% identity to said sequences.

This invention provides specific binding molecules, including TCR CDR and framework regions, which bind to the HLA-A11 restricted peptide VVVGADGVGK (SEQ ID No.1). Said specific binding molecules have particularly desirable therapeutic properties for the treatment of cancer.

The specific binding molecules or binding fragments thereof include TCR variable domains, which may correspond to those from a native TCR, or more preferably the TCR variable domains may be engineered. Native TCR variable domains may also be referred to as wild-type, natural, parental, unmutated or scaffold domains. The specific binding molecules or binding fragments can be used to produce molecules with ideal therapeutic properties such as supra-physiological affinity for target, long binding half-life, high specificity for target and good stability. The invention also includes bispecific, or bifunctional, or fusion, molecules that incorporate specific binding molecules or binding fragments thereof and a T cell redirecting moiety. Such molecules can mediate a potent and specific response against cancer cells by re-directing and activating a polyclonal T-cell response. Furthermore, the use of specific binding molecules with supra-physiological affinity facilitates recognition and clearance of cancer cells presenting low levels of peptide-HLA. Alternatively, the specific binding molecules or binding fragments may be fused to other therapeutic agents, and or diagnostic agents, and or incorporated in to engineered T cells for adoptive therapy.

The TCR domain sequences may be defined with reference to IMGT nomenclature which is widely known and accessible to those working in the TCR field. For example, see: LeFranc and LeFranc, (2001). “T cell Receptor Factsbook”, Academic Press; Lefranc, (2011), Cold Spring Harb Protoc 2011(6): 595-603; Lefranc, (2001), Curr Protoc Immunol Appendix 1: Appendix 10O; and Lefranc, (2003), Leukemia 17(1): 260-266. Briefly, αβ TCRs consist of two disulphide linked chains. Each chain (alpha and beta) is generally regarded as having two domains, namely a variable and a constant domain. A short joining region connects the variable and constant domains and is typically considered part of the alpha variable region. Additionally, the beta chain usually contains a short diversity region next to the joining region, which is also typically considered part of the beta variable region. The variable domain of each chain is located N-terminally and comprises three Complementarity Determining Regions (CDRs) embedded in a framework sequence (FR). The CDRs comprise the recognition site for peptide-MHC binding. There are several genes coding for alpha chain variable (Vα) regions and several genes coding for beta chain variable (Vβ) regions, which are distinguished by their framework, CDR1 and CDR2 sequences, and by a partly defined CDR3 sequence. The Vα and Vβ genes are referred to in IMGT nomenclature by the prefix TRAV and TRBV respectively (Folch and Lefranc, (2000), Exp Clin Immunogenet 17(1): 42-54; Scaviner and Lefranc, (2000), Exp Clin Immunogenet 17(2): 83-96; LeFranc and LeFranc, (2001), “T cell Receptor Factsbook”, Academic Press). Likewise there are several joining or J genes, termed TRAJ or TRBJ, for the alpha and beta chain respectively, and for the beta chain, a diversity or D gene termed TRBD (Folch and Lefranc, (2000), Exp Clin Immunogenet 17(2): 107-114; Scaviner and Lefranc, (2000), Exp Clin Immunogenet 17(2): 97-106; LeFranc and LeFranc, (2001), “T cell Receptor Factsbook”, Academic Press). The huge diversity of T cell receptor chains results from combinatorial rearrangements between the various V, J and D genes, which include allelic variants, and junctional diversity (Arstila, et al., (1999), Science 286(5441): 958-961; Robins et al., (2009), Blood 114(19): 4099-4107.) The constant, or C, regions of TCR alpha and beta chains are referred to as TRAC and TRBC respectively (Lefranc, (2001), Curr Protoc Immunol Appendix 1: Appendix 10).

As used herein, the term “specific binding molecule” refers to a molecule capable of binding to a target antigen. Such molecules may adopt a number of different formats as discussed herein. Furthermore, fragments of the specific binding molecules of the invention are also envisioned. A fragment refers to a portion of the specific binding molecule that retains binding to the target antigen. The term ‘mutations’ encompasses substitutions, insertions and deletions. Mutations to a native (also referred to as parental, natural, unmutated, wild type, or scaffold) specific binding molecule may confer beneficial therapeutic properties, such as high affinity, high specificity and high potency; for example, mutations may that include those that increase the binding affinity (k_{D}) and/or binding half-life (t_{½}) of the specific binding molecule to the VVVGADGVGK-HLA-A*11 complex.

The alpha chain framework regions FR1, FR2, and FR3 may comprise amino acid sequences corresponding to a TRAV19*01 chain and / or the beta chain framework regions FR1, FR2 and FR3, may comprise amino acid sequences corresponding to those of a TRBV6-⅔*01 chain.

The FR4 region may comprise the joining region of the alpha and beta variable chains (TRAJ and TRBJ, respectively). The TRAJ region may comprise amino acid sequences corresponding to those of TRAJ28*01. The TRBJ region may comprise amino acid sequences corresponding to those of TRBJ1-6*02.

In the TCR alpha chain variable region, there may be at least one mutation. There may be one or two or three or four or five or six or seven or eight or nine or ten or eleven or twelve or thirteen or fourteen or fifteen or sixteen or seventeen or more, mutations in the alpha chain CDRs (i.e. in total across all three CDRs). For example there may be 17 mutations or there may be 10 mutations in the alpha chain CDRs. One or more of said mutations may be selected from the following mutations, with reference to the numbering of SEQ ID NO: 2:

T31A, R51Q, N52P, S53W, F54W, D55G, E56S, Q57S, N58R, E59G, L94M, G96V, S98D,
G99S or G99M, A100R or A100E or A100D, S102H, L105F

Thus, there may be any or all of the mutations listed above, optionally in combination with other mutations

The mutated alpha chain CDRs may comprise one of the following groups of mutations (with reference to the numbering of SEQ ID NO: 2):
Group 1: T31A, R51Q, N52P, S53W, F54W, D55G, E56S, Q57S, N58R, E59G, L94M, G96V, S98D, G99S, A100R, S102H, L105F
Group 2: T31A, R51Q, N52P, S53W, F54W, D55G, E56S, Q57S, N58R, E59G, L94M, G96V, S98D, G99M, A100E, S102H, L105F
Group 3: T31A, R51Q, N52P, S53W, F54W, D55G, L94M, G96V, A100D, L105F

The alpha chain CDR1 may comprise the following sequence

TRDTTYY (SEQ ID No: 32)

TRDTAYY (SEQ ID No: 38)

The alpha chain CDR2 may comprise the following sequence

RNSFDEQNE (SEQ ID No: 33)

QPWWGSSRG (SEQ ID No: 39)

QPWWGEQNE (SEQ ID No: 40)

The alpha chain CDR3 may comprise the following sequence

CALSGPSGAGSYQLTF(SEQ ID No: 34)

CAMSVPDSRGHYQFTF (SEQ ID No: 41)

CAMSVPDMEGHYQFTF (SEQ ID No: 42)

CAMSVPSGDGSYQFTF (SEQ ID No: 43)

For example, in the mutated alpha chain CDR1 is TRDTAYY, CDR2 is QPWWGSSRG and CDR3 is CAMSVPDSRGHYQFTF. Alternatively, CDR1 is TRDTAYY, CDR2 is QPWWGSSRG and CDR3 is CAMSVPDMEGHYQFTF. Alternatively, CDR1 is TRDTAYY CDR2 is QPWWGEQNE and CDR3 is CAMSVPSGDGSYQFTF.

The mutated alpha chain may be paired with any beta chain.

In the TCR beta chain variable region, there may be at least one mutation. There may be one or two or three or four or five or six or seven or more, mutations in the beta chain CDRs (i.e. in total across all three CDRs). For example there may be 5 mutations or there may be 7 mutations in the beta chain CDRs. One or more of said mutations may be selected from the following mutations with reference to the numbering of SEQ ID NO: 3

V50G, G51W, E52G, G53K, T54D, S94K, Y95V

Thus, there may be any or all of the mutations listed above, optionally in combination with other mutations

The beta chain CDRs may comprise one of the following groups of mutations (with reference to the numbering of SEQ ID NO: 3):
Group 1: V50G, G51W, E52G, G53K, T54D, S94K, Y95V
Group 2: V50G, G51W, E52G, G53K, T54D,

The beta chain CDR1 may comprise the following sequence

MNHEY (SEQ ID No: 35)

The beta chain CDR2 may comprise the following sequence

SVGEGT (SEQ ID No: 36)

SGWGKD (SEQ ID No: 44)

The beta chain CDR3 may comprise the following sequence

CASSYGPGQHNSPLHF (SEQ ID No: 37)

CASKVGPGQHNSPLHF (SEQ ID No: 45)

For example, in the mutated beta chain CDR1 is MNHEY, CDR2 is SGWGKD and CDR3 is CASKVGPGQHNSPLHF. Alternatively, CDR1 is MNHEY, CDR2 is SGWGKD and CDR3 is CASSYGPGQHNSPLHF.

The mutated beta chain may be paired with any alpha chain.

Preferred pairing of alpha and beta chains comprise the following CDR sequences

In the alpha chain CDR1 is TRDTAYY, CDR2 is QPWWGSSRG and CDR3 is CAMSVPDSRGHYQFTF, and in the beta chain CDR1 is MNHEY, CDR2 is SGWGKD and CDR3 is CASKVGPGQHNSPLHF

In the alpha chain CDR1 is TRDTAYY, CDR2 is QPWWGSSRG and CDR3 is CAMSVPDMEGHYQFTF, and in the beta chain CDR1 is MNHEY, CDR2 is SGWGKD and CDR3 is CASSYGPGQHNSPLHF

In the alpha chain CDR1 is TRDTAYY CDR2 is QPWWGEQNE and CDR3 is CAMSVPSGDGSYQFTF and in the beta chain CDR1 is MNHEY, CDR2 is SGWGKD and CDR3 is CASSYGPGQHNSPLHF

Mutation(s) within the CDRs preferably improve the binding affinity or specificity of the specific binding molecule to the VWGADGVGK-HLA-A*11 complex, but may additionally or alternatively confer other advantages such as improved stability in an isolated form or improved potency when fused to an immune effector. Mutations at one or more positions may additionally or alternatively affect the interaction of an adjacent position with the cognate pMHC complex, for example by providing a more favourable angle for interaction. Mutations may include those that are result in a reduction in nonspecific binding, i.e. a reduction in binding to alternative antigens relative to VVVGADGVGK-HLA-A*11. Mutations may include those that increase efficacy of folding and/or stability and/or manufacturability. Some mutations may contribute to each of these characteristics; others may contribute to affinity but not to specificity, for example, or to specificity but not to affinity etc.

Typically, at least 5, at least 10, at least 15, or more CDR mutations in total are needed to obtain specific binding molecules with pM affinity for target antigen. At least 5, at least 10 or at least 15 CDR mutations in total may be needed to obtain specific binding molecules with pM affinity for target antigen. Specific binding molecules with pM affinity for target antigen are especially suitable as soluble therapeutics. Specific binding molecules for use in adoptive therapy applications may have lower affinity for target antigen and thus fewer CDR mutations, for example, up to 1, up to 2, up to 5, or more CDR mutations in total. In some cases, it may be possible to take a specific binding molecules with pM affinity and produce a lower affinity molecule by reverting one or more of the CDR mutations back to the original native residue. In some cases the native (also referred to as unmutated) specific binding molecule may have a sufficiently high affinity for target antigen without the need for mutation. It has been noted that the specific binding molecules of the present invention in their native form have advantageously therapeutic properties, including high specificity. Without wishing to be bound by any particular theory, the present inventors consider that the ability of the molecules of the invention to discriminate between WT and mutant Kras peptide is at least in part due to the different confirmation adopted by the mutant peptide when bound to HLA.

Mutations may additionally, or alternatively, be made outside of the CDRs, within the framework regions; such mutations may results in improved therapeutic properties, such as improved binding, and/or specificity, and/or stability, and/or the yield of a purified soluble form of the specific binding molecule. For example, the specific binding molecule of the invention may, additionally or alternatively, comprise one or more mutations at the N terminus of FR1, of one of both chains, in order to improve the efficiency of N-terminal methionine cleavage. The removal of an N-terminal initiation methionine is often crucial for the function and stability of proteins. Inefficient cleavage may be detrimental for a therapeutic, since it may result in a heterogeneous protein product, and or the presence of the initiation methionine may be immunogenic in humans. In some case an initiation methionine may be present in the specific binding molecules of the invention.

Preferably, the α chain variable domain of the specific binding molecule of the invention may comprise respective framework amino acid sequences that have at least 90%, at least 91%, at least 92%, at least 93%, at least 94%, at least 95%, at least 96%, at least 97%, at least 98 % or at least 99% identity to the framework amino acid residues 1-26, 34-50, 60-91, 108-117 of SEQ ID NO: 2. The beta chain variable domain of the specific binding molecule of the invention may comprise respective framework amino acid sequences that have at least 90%, at least 91%, at least 92%, at least 93%, at least 94%, at least 95%, at least 96%, at least 97%, at least 98 % or at least 99% identity to the framework amino acid residues 1-26, 32-48, 55-90, 106-115 of SEQ ID NO: 3. Alternatively, the stated percentage identity may be over the framework sequences when considered as a whole.

The alpha chain variable domain may comprise any one of the amino acid sequences of SEQ ID NOs: 4-6 (shown in FIG. 2) and the beta chain variable domain may comprise any one of the amino acid sequences of SEQ ID NOs: 7-8 (shown in FIG. 3).

For example, the specific binding molecule may comprise the following alpha and beta chain pairs.

| | |
|---|---|
| Alpha chain variable domain | Beta chain variable domain |
| SEQ ID No 4 | SEQ ID No 7 |
| SEQ ID No 5 | SEQ ID No 8 |
| SEQ ID No 6 | SEQ ID No 8 |

A preferred TCR chain pairing is SEQ ID NO: 4 and SEQ ID NO: 7.

Within the scope of the invention are phenotypically silent variants of any specific binding molecule of the invention disclosed herein. As used herein the term “phenotypically silent variants” is understood to refer to a specific binding molecule with a TCR variable domain which incorporates one or more further amino acid changes, including substitutions, insertions and deletions, in addition to those set out above, which specific binding molecule has a similar phenotype to the corresponding specific binding molecule without said change(s). For the purposes of this application, specific binding molecule phenotype comprises binding affinity (K_{D} and/or binding half-life) and specificity. Preferably, the phenotype for a soluble specific binding molecule associated with an immune effector includes potency of immune activation and purification yield, in addition to binding affinity and specificity. A phenotypically silent variant may have a K_{D} and/or binding half-life for the VWGADGVGK-HLA-A*11 complex within 50%, or more preferably within 30%, 25% or 20%, of the measured K_{D} and/or binding half-life of the corresponding specific binding molecule without said change(s), when measured under identical conditions (for example at 25° C. and/or on the same SPR chip). Suitable conditions are further provided in Examples 1 and 2.

Furthermore, a phenotypically silent variant may retain the same, or sustainably the same, therapeutic window between binding to the VVVGADGVGK-HLA-A*11 complex and binding to the WT KRAS peptide, and or binding to one or more additional off-target peptide-HLA complex. A phenotypically silent variant may retain the same, or sustainably the same, therapeutic window between potency of immune cell activation in response to cells presenting to the VVVGADGVGK-HLA-A*11 complex and the WT KRAS peptide, and or cells presenting one or more additional off-target peptide-HLA complex. The therapeutic window may be calculated based on lowest effective concentrations (“LOEL”) observed for normal cells and the tumor cell line. The therapeutic window may be at least 100 fold difference, at least 1000 fold difference, or more. A phenotypic variant may share the same, or substantially the same recognition motif as determined by sequential mutagenesis techniques discussed further below.

As is known to those skilled in the art, it may be possible to produce specific binding molecules that incorporate changes in the variable domains thereof compared to those detailed above without altering the affinity or specificity of the interaction with the VVVGADGVGK-HLA-A*11 complex, and or any other functional characteristics. In particular, such silent mutations may be incorporated within parts of the sequence that are known not to be directly involved in antigen binding (e.g. the framework regions and or parts of the CDRs that do not contact the antigen). Such variants are included in the scope of this invention.

As will be obvious to those skilled in the art, it may be possible to truncate, or extend, the sequences provided at the C-terminus and/or N-terminus thereof, by 1, 2, 3, 4, 5 or more residues, without substantially affecting the functional characteristics of the specific binding molecule. The sequences provided at the C-terminus and/or N-terminus thereof may be truncated or extended by 1, 2, 3, 4 or 5 residues. All such variants are encompassed by the present invention.

Phenotypically silent variants may contain one or more conservative substitutions and/or one or more tolerated substitutions. By tolerated substitutions it is meant those substitutions which do not fall under the definition of conservative as provided below but are nonetheless phenotypically silent. The skilled person is aware that various amino acids have similar properties and thus are ‘conservative’. One or more such amino acids of a protein, polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, it is contemplated herein that the methyl group on an alanine may be replaced with an ethyl group, and/or that minor changes may be made to the peptide backbone. Whether or not natural or synthetic amino acids are used, it is preferred that only L- amino acids are present.

Substitutions of this nature are often referred to as “conservative” or “semi-conservative” amino acid substitutions. The present invention therefore extends to use of a specific binding molecule comprising any of the amino acid sequence described above but with one or more conservative substitutions and or one or more tolerated substitutions in the sequence, such that the amino acid sequence of the specific binding molecule has at least 90% identity, such as 90%, 91%, 92%, 93%, 94%, 95%, 96%, 97%, 98%, 99% or 100% identity, to the specific binding molecule comprising amino acids 1-117 of SEQ ID NOs: 2, 4-6, and/or amino acids 1-115 of SEQ ID NOs: 3, 7-8.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs.

Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)) SIM - Alignment Tool for protein sequences (Xiaoquin Huang and Webb Miller: “A Time-Efficient, Linear-Space Local Similarity Algorithm” Advances in Applied Mathematics, vol. 12 (1991), pp. 337-357).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity = number of identical positions/total number of positions × 100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The BLASTn and BLASTp programs of Altschul, et al. (1990) J. Mol. Biol. 215:403-410 have incorporated such an algorithm. Determination of percent identity between two nucleotide sequences can be performed with the BLASTn program. Determination of percent identity between two protein sequences can be performed with the BLASTp program. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (ld.). When utilising BLAST, Gapped BLAST, and PSI-Blast programs, the default parameters of the respective programs (e.g., BLASTp and BLASTp) can be used. See http://www.ncbi.nlm.nih.gov. Default general parameters may include for example, Word Size = 3, Expect Threshold = 10. Parameters may be selected to automatically adjust for short input sequences. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10 :3-5; and FASTA described in Pearson and Lipman (1988) Proc. Natl. Acad. Sci. 85:2444-8. Within FASTA, ktup is a control option that sets the sensitivity and speed of the search. For the purposes of evaluating percent identity in the present disclosure, BLASTp with the default parameters is used as the comparison methodology. In addition, when the recited percent identity provides a non-whole number value for amino acids (i.e., a sequence of 25 amino acids having 90% sequence identity provides a value of “22.5”, the obtained value is rounded down to the next whole number, thus “22”). Accordingly, in the example provided, a sequence having 22 matches out of 25 amino acids is within 90% sequence identity.

Mutations, including conservative and tolerated substitutions, insertions and deletions, may be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning — A Laboratory Manual (3^{rd} Ed.) CSHL Press. Further information on ligation independent cloning (LIC) procedures can be found in Rashtchian, (1995) Curr Opin Biotechnol 6(1): 30-6. The TCR sequences provided by the invention may be obtained from solid state synthesis, or any other appropriate method known in the art.

The specific binding molecules of the invention have the property of binding the VVVGADGVGK-HLA-A*11 complex. Specific binding molecules of the invention demonstrate a high degree of specificity for VWGADGVGK-HLA-A*11 complex and are thus particularly suitable for therapeutic use. Specificity in the context of specific binding molecules of the invention relates to their ability to recognise target cells that are antigen positive, whilst having minimal ability to recognise target cells that are antigen negative. Antigen positive cells are those that have been determined to express mutant KRAS and or those that have been determined to present the WVGADGVGK-HLA-A*11 complex. The specific binding molecules of the invention may bind the complex of target peptide when bound to one of more HLA-A*11 subtypes, for example the specific binding molecules of the invention may bind the complex of the target peptide when bound to HLA-A*1101 and or the specific binding molecules of the invention may bind the complex of the target peptide when bound to HLA-A*1102.

Specificity can be measured in vitro, for example, in cellular assays such as those described in Examples 3 and 4. To test specificity, the specific binding molecules may be in soluble form and associated with an immune effector, and/or may be expressed on the surface of cells, such as T cells. Specificity may be determined by measuring the level of T cell activation in the presence of antigen positive and antigen negative target cells as defined above. Minimal recognition of antigen negative target cells is defined as a level of T cell activation of less than 20%, preferably less than 10%, preferably less than 5%, and more preferably less than 1%, of the level produced in the presence of antigen positive target cells, when measured under the same conditions and at a therapeutically relevant TCR concentration. For soluble TCRs associated with an immune effector, a therapeutically relevant concentration may be defined as a concentration of 10⁻⁹ M or below, and/or a concentration of up to 100, preferably up to 1000, fold greater than the corresponding EC50 or IC50 value. Preferably, for soluble specific binding molecules associated with an immune effector there is at least a 100 fold, at least 1000 fold, at least 10000 fold difference in EC50 or IC50 value between T cell activation against antigen positive cells relative to antigen negative cells - this difference may be referred to as a therapeutic window. Additionally or alternatively the therapeutic window may be calculated based on lowest effective concentrations (“LOEL”) observed for normal cells and the tumor cell line. Antigen positive cells may be obtained by peptide-pulsing using a suitable peptide concentration to obtain a level of antigen presentation comparable to cancer cells (for example, 10⁻⁹ M peptide, as described in Bossi et al., (2013) Oncoimmunol. 1;2 (11) :e26840) or, they may naturally present said peptide. Preferably, both antigen positive and antigen negative cells are human cells. Preferably antigen positive cells are human cancer cells. Antigen negative cells preferably include those derived from healthy human tissues. Antigen negative cells may include those that express and or present wild-type KRAS peptide.

Specificity may additionally, or alternatively, relate to the ability of a specific binding molecule to bind to VWGADGVGK-HLA-A*11 complex and not to a panel of alternative peptide-HLA complexes or the WT KRAS peptide. This may, for example, be determined by the Biacore method of Examples 1 and 2. Said panel may contain at least 5, and preferably at least 10, alternative peptide-HLA complexes. The alternative peptides may share a low level of sequence identity with SEQ ID NO: 1 and may be naturally presented. Alternative peptides are preferably derived from proteins expressed in healthy human tissues. Binding of the specific binding molecule to the WVGADGVGK-HLA-A*11 complex may be at least 2 fold greater than to other naturally-presented peptide HLA complexes, more preferably at least 10 fold, or at least 100 fold or at least 1000 fold greater or at least 3000 fold greater.

An alternative or additional approach to determine specific binding molecule specificity may be to identify the peptide recognition motif of the specific binding molecule using sequential mutagenesis, e.g. alanine scanning, of the target peptide. Residues that form part of the binding motif are those that are not permissible to substitution. Non-permissible substitutions may be defined as those peptide positions in which the binding affinity of the specific binding molecule is reduced by at least 50%, or at least 80%, relative to the binding affinity for the non-mutated peptide. Such an approach is further described in Cameron et al., (2013), Sci Transl Med. 2013 Aug 7; 5 (197): 197ra103 and WO2014096803 in connection with TCRs, though it will be appreciated that such methods can also be applied to the specific binding molecules of the present invention. Specific binding molecule specificity in this case may be determined by identifying alternative motif containing peptides, particularly alternative motif containing peptides in the human proteome, and testing these peptides for binding to the specific binding molecule. Binding of the specific binding molecule to one or more alternative peptides may indicate a lack of specificity. In this case further testing of specific binding molecule specificity via cellular assays may be required. A low tolerance for (alanine) substitutions in the central part of the peptide indicates that the specific binding molecule has a high specificity and therefore presents a low risk for cross-reactivity with alternative peptides.

Specific binding molecules of the invention may have an ideal safety profile for use as therapeutic reagents. In this case the specific binding molecules may be in soluble form and may preferably be fused to an immune effector. Suitable immune effectors include but are not limited to, cytokines, such as IL-2 and IFN-y; superantigens and mutants thereof; chemokines such as IL-8, platelet factor 4, melanoma growth stimulatory protein; antibodies and antibody like scaffolds, including fragments, derivatives and variants thereof that bind to antigens on immune cells such as T cells or NK cell (e.g. anti-CD3, anti-CD28 or anti-CD16); and Fc receptor or complement activators. An ideal safety profile means that in addition to demonstrating good specificity, the specific binding molecules of the invention may have passed further preclinical safety tests. Examples of such tests include whole blood assays to confirm minimal cytokine release in the presence of whole blood and thus low risk of causing a potential cytokine release syndrome in vivo, and alloreactivity tests to confirm low potential for recognition of alternative HLA types.

Specific binding molecules of the invention may be amenable to high yield purification, particularly specific binding molecules in soluble format. Yield may be determined based on the amount of correctly folded material obtained at the end of the purification process relative to the original culture volume. High yield typically means greater than 1 mg/L, or greater than 2 mg/L, or more preferably greater than 3 mg/L, or greater than 4 mg/L or greater than 5 mg/L, or higher yield.

Mutated specific binding molecules of the invention preferably have a K_{D} for the WVGADGVGK-HLA-A*11 complex of greater than (i.e. stronger than) the native TCR (also referred to as the non-mutated, or scaffold TCR), for example in the range of 1 pM to 1 µM. In one aspect, specific binding molecules of the invention have a K_{D} for the complex of from about (i.e. +/- 10%) 1 pM to about 400 nM, from about 1 pM to about 1000 pM, from about 1 pM to about 500 pM, from about 1 pM to about 100 pM. Said specific binding molecules may additionally, or alternatively, have a binding half-life (T½) for the complex in the range of from about 1 min to about 60 h, from about 20 min to about 50 h, or from about 2 h to about 35 h, or from about 4 hours to about 20 hours. Preferably, specific binding molecules of the invention have a K_{D} for the WVGADGVGK-HLA-A *11 complex of from about 1 pM to about 200 pM and/or a binding half-life from about 4 h to about 20 h. Such high-affinity is preferable for specific binding molecules in soluble format when associated with therapeutic agents and/or detectable labels.

In another aspect, mutated specific binding molecules of the invention may have a K_{D} for the complex of from about 50 nM to about 200 µM, or from about 100 nM to about 2 µM and/or a binding half-life for the complex of from about 3 sec to about 12 min. Such specific binding molecules may be preferable for adoptive therapy applications.

Methods to determine binding affinity (inversely proportional to the equilibrium constant K_{D}) and binding half life (expressed as T½) are known to those skilled in the art. In a preferred embodiment, binding affinity and binding half-life are determined using Surface Plasmon Resonance (SPR) or Bio-Layer Interferometry (BLI), for example using a BlAcore instrument or Octet instrument, respectively. A preferred method is provided in Examples 1 and 2. It will be appreciated that doubling the affinity of a specific binding molecule results in halving the K_{D}. T½ is calculated as In2 divided by the off-rate (k_{off}). Therefore, doubling of T½ results in a halving in k_{off}. K_{D} and k_{off} values for TCRs are usually measured for soluble forms of the TCR, i.e. those forms which are truncated to remove cytoplasmic and transmembrane domain residues (including single chain TCRs and or TCR incorporating a non-native disulphide bond or other dimerization domain). To account for variation between independent measurements, and particularly for interactions with dissociation times in excess of 20 hours, the binding affinity and or binding half-life of a given specific binding molecule may be measured several times, for example 3 or more times, using the same assay protocol, and an average of the results taken. To compare binding data between two samples (i.e. two different specific binding molecules and or two preparations of the same specific binding molecule) it is preferable that measurements are made using the same assay conditions (e.g. temperature), such as those described in Example 1 and 2.

Certain preferred mutated specific binding molecules of the invention have a binding affinity for, and/or a binding half-life for, the VVVGADGVGK-HLA-A*11 complex that is substantially higher than that of the native TCR. Increasing the binding affinity of a native TCR may reduce the specificity of the TCR for its peptide-MHC ligand, and this is demonstrated in Zhao et al., (2007) J.Immunol, 179:9, 5845-5854. However, such mutated specific binding molecules of the invention remain specific for the VVVGADGVGK-HLA-A*11 complex, despite having substantially higher binding affinity than the native TCR.

Certain preferred mutated specific binding molecules of the invention are able to generate a highly potent T cell response in vitro against antigen positive cells, in particular those cells presenting low levels of antigen (i.e. in the order of 5-100). Such specific binding molecules may be in soluble form and linked to an immune effector such as an anti-CD3 antibody. The T cell response that is measured may be the release of T cell activation markers such as Interferon y or Granzyme B, or target cell killing, or other measure of T cell activation, such as T cell proliferation. Preferably a highly potent response is one with EC50 or IC50 value in the pM range, for example, 1000 pM or lower, or 500 pM or lower, or 200 pM or lower.

Specific binding molecules of the invention may comprise TCR variable domains. Preferably the TCR variable domains comprise a heterodimer of alpha and beta chains. Alternatively, the TCR variable domains may comprise a heterodimer of gamma and delta chains. In some cases, the specific binding molecules of the invention may comprise homodimers of TCR variable domains such as αα or ββ homodimers (or yy or δδ homodimers).

In the specific binding molecules of the invention the variable domains and where present the constant domains, and or any other domains, may be organised in any suitable format/arrangement. Examples of such arrangements are well known in the antibody art. The skilled person is aware of the similarities between antibodies and TCRs and could apply such arrangements to TCR variable and constant domains (Brinkman et al., MAbs. 2017 Feb-Mar; 9(2): 182-212). For example, the variable domains may be arranged in monoclonal TCR format, in which the two chains are linked by a disulphide bond, either within the constant domains or variable domains, or in which the variable domains are fused to one or more dimerization domains. Alternatively the variable domains may be arranged in single chain format in the present or absence of one or more constant domains, or the variable domains may be arranged in diabody format.

Specific binding molecules of the invention may comprise at least one TCR constant domain or fragment thereof, for example an alpha chain TRAC constant domain and/or a beta chain TRBC1 or TRBC2 constant domain. As will be appreciated by those skilled in the art the term TRAC and TRBC½ also encompasses natural polymorphic variants, for example N to K at position 4 of TRAC (Bragado et al International immunology. 1994 Feb;6(2):223-30).

Where present, one or both of the constant domains may contain mutations, substitutions or deletions relative to native constant domain sequences. The constant domains may be truncated, i.e. having no transmembrane or cytoplasmic domains. Alternatively the constant domains may be full-length by which it is meant that extracellular, transmembrane and cytoplasmic domains are all present. The TRAC and TRBC domain sequences may be modified by truncation or substitution to delete the native disulphide bond between Cys4 of exon 2 of TRAC and Cys2 of exon 2 of TRBC1 or TRBC2. The alpha and/or beta chain constant domain sequence(s) may have an introduced disulphide bond between residues of the respective constant domains, as described, for example, in WO 03/020763. Preferably the alpha and beta constant domains may be modified by substitution of cysteine residues at position Thr 48 of TRAC and position Ser 57 of TRBC1 or TRBC2, the said cysteines forming a non-natural disulphide bond between the alpha and beta constant domains of the TCR. TRBC1 or TRBC2 may additionally include a cysteine to alanine mutation at position 75 of the constant domain and an asparagine to aspartic acid mutation at position 89 of the constant domain. One or both of the extracellular constant domains present in an αβ heterodimer of the invention may be further truncated at the C terminus or C termini, for example by up to 15, or up to 10, or up to 8 or fewer amino acids. One or both of the extracellular constant domains present in an αβ heterodimer of the invention may be truncated at the C terminus or C termini by, for example, up to 15, or up to 10 or up to 8 amino acids. The C terminus of the alpha chain extracellular constant domain may be truncated by 8 amino acids.

Alternatively, rather than full-length or truncated constant domains there may be no TCR constant domains. Accordingly, the specific binding molecule of the invention may be comprised of the variable domains of the TCR alpha and beta chains, optionally with additional domains as described herein. Additional domains include but are not limited to immune effector domains (such as antibody domains), Fc domains or albumin binding domains, therapeutic agents or detectable labels.

Single chain formats include, but are not limited to, αβ TCR polypeptides of the Vα-L-Vβ, Vβ-L-Vα, Vα-Cα-L-Vβ, Vα-L-Vβ-Cβ, or Vα-Cα-L-Vβ-Cβ types, wherein Vα and Vβ are TCR α and β variable regions respectively, Cα and Cβ are TCR α and β constant regions respectively, and L is a linker sequence (Weidanz et al., (1998) J Immunol Methods. Dec 1;221(1-2):59-76; Epel et al., (2002), Cancer Immunol Immunother. Nov;51(10):565-73; WO 2004/033685; WO9918129). Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length, The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used multi-domain binding molecules of the invention include, but are not limited to: GGGGS (SEQ ID No: 18), GGGSG (SEQ ID No: 19), GGSGG (SEQ ID No: 20), GSGGG (SEQ ID No: 21), GSGGGP (SEQ ID No: 22), GGEPS (SEQ ID No: 23), GGEGGGP (SEQ ID No: 24), and GGEGGGSEGGGS (SEQ ID No: 25) (as described in WO2010/133828) and GGGSGGGG (SEQ ID NO: 26). Additional linkers may include sequences having one or more of the following sequence motifs: GGGS (SEQ ID NO: 27), GGGGS (SEQ ID NO: 28), TVLRT (SEQ ID NO: 29), TVSSAS (SEQ ID NO: 30) and TVLSSAS (SEQ ID NO: 31). Where present, one or both of the constant domains may be full length, or they may be truncated and/or contain mutations as described above. Preferably single chain TCRs are soluble. In certain embodiments single chain TCRs of the invention may have an introduced disulphide bond between residues of the respective constant domains, as described in WO 2004/033685. Single chain TCRs are further described in WO2004/033685; WO98/39482; WO01/62908; Weidanz et al. (1998) J Immunol Methods 221(1-2): 59-76; Hoo et al. (1992) Proc Natl Acad Sci U S A 89(10): 4759-4763; Schodin (1996) Mol Immunol 33(9): 819-829).

The TCR variable domains may be arranged in diabody format. In the diabody format two single chain fragments dimerize in a head-to-tail orientation resulting in a compact molecule with a molecular mass similar to tandem scFv (~50 kDa).

The invention also includes particles displaying specific binding molecules of the invention and the inclusion of said particles within a library of particles. Such particles include but are not limited to phage, yeast cells, ribosomes, or mammalian cells. Method of producing such particles and libraries are known in the art (for example see WO2004/044004; WO01/48145, Chervin et al. (2008) J. Immuno. Methods 339.2: 175-184).

Specific binding molecules of the invention are useful for delivering detectable labels or therapeutic agents to antigen presenting cells and tissues containing antigen presenting cells. They may therefore be associated (covalently or otherwise) with a detectable label (for diagnostic purposes wherein the specific binding molecule is used to detect the presence of cells presenting the cognate antigen); and or a therapeutic agent, including immune effectors; and or a pharmacokinetic (PK) modifying moiety.

Examples of PK modifying moieties include, but are not limited to, PEG (Dozier et al., (2015) Int J Mol Sci. Oct 28;16(10):25831-64 and Jevsevar et al., (2010) Biotechnol J.Jan;5(1):113-28), PASylation (Schlapschy et al., (2013) Protein Eng Des Sel. Aug;26(8):489-501), albumin, and albumin binding domains, (Dennis et al., (2002) J Biol Chem. Sep 20;277(38):35035-43), and/or unstructured polypeptides (Schellenberger et al., (2009) Nat Biotechnol. Dec;27(12):1186-90). Further PK modifying moieties include antibody Fc fragments. PK modifying moieties may serve to extend the in vivo half-life of specific binding molecules of the invention.

Where an immunoglobulin Fc domain is used, it may be any antibody Fc region. The Fc region is the tail region of an antibody that interacts with cell surface Fc receptors and some proteins of the complement system. The Fc region typically comprises two polypeptide chains both having two or three heavy chain constant domains (termed CH2, CH3 and CH4), and a hinge region. The two chains being linked by disulphide bonds within the hinge region. Fc domains from immunoglobulin subclasses IgG1, IgG2 and IgG4 bind to and undergo FcRn mediated recycling, affording a long circulatory half-life (3 - 4 weeks). The interaction of IgG with FcRn has been localized in the Fc region covering parts of the CH2 and CH3 domain. Preferred immunoglobulin Fc for use in the present invention include, but are not limited to Fc domains from IgG1 or IgG4. Preferably the Fc domain is derived from human sequences. The Fc region may also preferably include KiH mutations which facilitate dimerization, as well as mutations to prevent interaction with activating receptors i.e. functionally silent molecules. The immunoglobulin Fc domain may be fused to the C or N terminus of the other domains (i.e., the TCR variable domains and / or TCR constant domains and/or immune effector domains), in any suitable order or configuration. The immunoglobulin Fc may be fused to one or more of the other domains (i.e., the TCR variable domains and / or TCR constant domains and /or an immune effector domains) via a linker. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length Examples of suitable linkers that may be used multi-domain binding molecules of the invention include, but are not limited to: GGGGS (SEQ ID No: 18), GGGSG (SEQ ID No: 19), GGSGG (SEQ ID No: 20), GSGGG (SEQ ID No: 21), GSGGGP (SEQ ID No: 22), GGEPS (SEQ ID No: 23), GGEGGGP (SEQ ID No: 24), and GGEGGGSEGGGS (SEQ ID No: 25) (as described in WO2010/133828) and GGGSGGGG (SEQ ID NO: 26). Additional linkers may include sequences having one or more of the following sequence motifs: GGGS (SEQ ID NO: 27), GGGGS (SEQ ID NO: 28), TVLRT (SEQ ID NO: 29), TVSSAS (SEQ ID NO: 30) and TVLSSAS (SEQ ID NO: 31). Where the immunoglobulin Fc is fused to the TCR, it may be fused to either the alpha or beta chains, with or without a linker. Furthermore, individual chains of the Fc may be fused to individual chains of the TCR.

Preferably the Fc region may be derived from the IgG1 or IgG4 subclass. The two chains may comprise CH2 and CH3 constant domains and all or part of a hinge region. The hinge region may correspond substantially or partially to a hinge region from IgG1, IgG2, IgG3 or IgG4. The hinge may comprise all or part of a core hinge domain and all or part of a lower hinge region. Preferably, the hinge region contains at least one disulphide bond linking the two chains.

The Fc region may comprise mutations relative to a WT sequence. Mutations include substitutions, insertions and deletions. Such mutations may be made for the purpose of introducing desirable therapeutic properties. For example, to facilitate heterodimersation, knobs into holes (KiH) mutations maybe engineered into the CH3 domain. In this case, one chain is engineered to contain a bulky protruding residue (i.e. the knob), such as Y, and the other is chain engineered to contain a complementary pocket (i.e. the hole). Suitable positions for KiH mutations are known in the art. Additionally or alternatively mutations may be introduced that abrogate or reduce binding to Fcy receptors and or increase binding to FcRn, and / or prevent Fab arm exchange, or remove protease sites. Additionally or alternatively mutations may be made to improve manufacturability for example to remove or alter glycosylation sites.

The PK modifying moiety may also be an albumin-binding domain, which may also act to extend half-life. As is known in the art, albumin has a long circulatory half-life of 19 days, due in part to its size, being above the renal threshold, and by its specific interaction and recycling via FcRn. Attachment to albumin is a well-known strategy to improve the circulatory half-life of a therapeutic molecule in vivo. Albumin may be attached non-covalently, through the use of a specific albumin binding domain, or covalently, by conjugation or direct genetic fusion. Examples of therapeutic molecules that have exploited attachment to albumin for improved half-life are given in Sleep et al., Biochim Biophys Acta. 2013 Dec;1830(12):5526-34.

The albumin-binding domain may be any moiety capable of binding to albumin, including any known albumin-binding moiety. Albumin binding domains may be selected from endogenous or exogenous ligands, small organic molecules, fatty acids, peptides and proteins that specifically bind albumin.

Examples of preferred albumin binding domains include short peptides, such as described in Dennis et al., J Biol Chem. 2002 Sep 20;277(38):35035-43 (for example the peptide QRLMEDICLPRWGCLWEDDF); proteins engineered to bind albumin such as antibodies, antibody fragments and antibody like scaffolds, for example Albudab® (O′Connor-Semmes et al., Clin Pharmacol Ther. 2014 Dec;96(6):704-12), commercially provided by GSK and Nanobody® (Van Roy et al., Arthritis Res Ther. 2015 May 20;17:135), commercially provided by Ablynx; and proteins based on albumin binding domains found in nature such as Streptococcal protein G Protein (Stork et al., Eng Des Sel. 2007 Nov;20(11):569-76), for example Albumod® commercially provided by Affibody Preferably, albumin is human serum albumin (HSA). The affinity of the albumin binding domain for human albumin may be in the range of picomolar to micromolar. Given the extremely high concentration of albumin in human serum (35-50 mg/ml, approximately 0.6 mM), it is calculated that substantially all of the albumin binding domains will be bound to albumin in vivo.

The albumin-binding moiety may be fused to the C or N terminus of the other domains (i.e., the TCR variable domains and / or TCR constant domains and/or immune effector domains), in any suitable order or configuration. The albumin-binding moiety may be fused to one or more of the other domains (i.e., the TCR variable domains and / or TCR constant domains and /or an immune effector domains) via a linker. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. The liker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used multi-domain binding molecules of the invention include, but are not limited to: GGGGS (SEQ ID No: 18), GGGSG (SEQ ID No: 19), GGSGG (SEQ ID No: 20), GSGGG (SEQ ID No: 21), GSGGGP (SEQ ID No: 22), GGEPS (SEQ ID No: 23), GGEGGGP (SEQ ID No: 24), and GGEGGGSEGGGS (SEQ ID No: 25) (as described in WO2010/133828) and GGGSGGGG (SEQ ID NO: 26). Additional linkers may include sequences having one or more of the following sequence motifs: GGGS (SEQ ID NO: 27), GGGGS (SEQ ID NO: 28), TVLRT (SEQ ID NO: 29), TVSSAS (SEQ ID NO: 30) and TVLSSAS (SEQ ID NO: 31). Where the albumin-binding moiety is linked to the TCR, it may be linked to either the alpha or beta chains, with or without a linker.

Detectable labels for diagnostic purposes include for instance, fluorescent labels, radiolabels, enzymes, nucleic acid probes and contrast reagents.

For some purposes, the specific binding molecules of the invention may be aggregated into a complex comprising several specific binding molecules to form a multivalent specific binding molecule complex. There are a number of human proteins that contain a multimerisation domain that may be used in the production of multivalent specific binding molecule complexes. For example the tetramerisation domain of p53 which has been utilised to produce tetramers of scFv antibody fragments which exhibited increased serum persistence and significantly reduced off-rate compared to the monomeric scFv fragment (Willuda et al. (2001) J. Biol. Chem. 276 (17) 14385-14392). Haemoglobin also has a tetramerisation domain that could be used for this kind of application. A multivalent specific binding molecule complex of the invention may have enhanced binding capability for the complex compared to a non-multimeric native (also referred to as parental, natural, unmutated wild type, or scaffold) T cell receptor heterodimer of the invention. Thus, multivalent complexes of specific binding molecules of the invention are also included within the invention. Such multivalent specific binding molecule complexes according to the invention are particularly useful for tracking or targeting cells presenting particular antigens in vitro or in vivo, and are also useful as intermediates for the production of further multivalent specific binding molecule complexes having such uses.

Therapeutic agents which may be associated with the specific binding molecules of the invention include immune-modulators and effectors, radioactive compounds, enzymes (perforin for example) or chemotherapeutic agents (cis-platin for example). To ensure that the therapeutic effects are exercised in the desired location the agent could be inside a liposome or other nanoparticle structure linked to the specific binding molecule so that the compound is released slowly. This will prevent damaging effects during the transport in the body and ensure that the agent has maximum effect after binding of the specific binding molecule to the relevant antigen presenting cells.

Examples of suitable therapeutic agents include, but are not limited to:
antibodies, or fragments thereof, including anti-T cell or NK cell determinant antibodies (e.g. anti-CD3, anti-CD28 or anti-CD16)
alternative protein scaffolds with antibody-like binding characteristics (e.g. DARPins)
immuno-stimulants, i.e. immune effector molecules which stimulate immune response. For example, cytokines such as IL-2 and IFN-y,
chemokines such as IL-8, platelet factor 4, melanoma growth stimulatory protein, etc.
activators of the complement pathway or Fc receptors
checkpoint inhibitors, such as those that target PD1 or PD-L1
small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate arbourate, auristatin E vincristine and doxorubicin
peptide cytotoxins, i.e. proteins or fragments thereof with the ability to kill mammalian cells.
For example, ricin, diphtheria toxin, pseudomonas bacterial exotoxin A, Dnase and Rnase;
radio-nuclides, i.e. unstable isotopes of elements which decay with the concurrent emission of one or more of α, or β particles, or γ rays. For example, iodine 131, rhenium 186, indium 111, yttrium 90, bismuth 210 and 213, actinium 225 and astatine 213; chelating agents may be used to facilitate the association of these radio-nuclides to TCRs, or multimers thereof;
superantigens and mutants thereof
peptide-HLA complex, wherein said peptide is derived from a common human pathogen, such as Epstein Barr Virus (EBV)
xenogeneic protein domains, allogeneic protein domains, viral/bacterial protein domains, viral/bacterial peptides

Preferred is a soluble specific binding molecule of the invention associated (usually by fusion to the Nor C-terminus of the alpha or beta chain, or both, in any suitable configuration) with an immune effector. The N terminus of the TCR may be linked to the C-terminus of the immune effector polypeptide.

A particularly preferred immune effector is an anti-CD3 antibody, or a functional fragment or variant of said anti-CD3 antibody. As used herein, the term “antibody” encompasses such fragments and variants. Examples of anti-CD3 antibodies include but are not limited to OKT3, UCHT-1, BMA-031 and 12F6. Antibody fragments and variants/analogues which are suitable for use in the compositions and methods described herein include minibodies, diabodies, Fab fragments, F(ab′)₂ fragments, dsFv and scFv fragments. Further examples encompassed within the term antibodies include Nanobodies™ (these constructs, marketed by Ablynx (Belgium), comprising synthetic single immunoglobulin variable heavy domain derived from a camelid (e.g. camel or llama) antibody), Domain Antibodies (Domantis, Belgium), comprising an affinity matured single immunoglobulin variable heavy domain or immunoglobulin variable light domain, and alternative protein scaffolds that exhibit antibody like binding characteristics, such as Affibodies (Affibody, Sweden), comprising engineered protein A scaffold, or Anticalins (Pieris, Germany), comprising engineered anticalins, or DARPins (Molecular Partners, Switzerland), comprising designed ankyrin repeat proteins.

Examples of preferred arrangements of fusion molecules include those described in WO2010133828 WO2019012138 and WO2019012141.

The specific binding molecule of the invention may comprise:
a first polypeptide chain which comprises the alpha chain variable domain and a first binding region of a variable domain of an antibody; and
a second polypeptide chain which comprises the beta chain variable domain and a second binding region of a variable domain of said antibody,
wherein the respective polypeptide chains associate such that the specific binding molecule is capable of simultaneously binding VVVGADGVGK (SEQ ID NO: 1) HLA-A*11 complex and an antigen of the antibody.

There is also provided herein a dual specificity polypeptide molecule selected from the group of molecules comprising a first polypeptide chain and a second polypeptide chain, wherein: the first polypeptide chain comprises a first binding region of a variable domain (VD1 ) of an antibody specifically binding to a cell surface antigen of a human immune effector cell, and
a first binding region of a variable domain (VR1 ) of a TCR specifically binding to an MHC-associated peptide epitope, and
a first linker (LINK1 ) connecting said domains;
the second polypeptide chain comprises a second binding region of a variable domain (VR2) of a TCR specifically binding to an MHC-associated peptide epitope, and
a second binding region of a variable domain (VD2) of an antibody specifically binding to a cell surface antigen of a human immune effector cell, and
a second linker (LINK2) connecting said domains;
wherein said first binding region (VD1 ) and said second binding region (VD2) associate to form a first binding site (VD1 )(VD2) that binds a cell surface antigen of a human immune effector cell;
said first binding region (VR1 ) and said second binding region (VR2) associate to form a second binding site (VR1 )(VR2) that binds said MHC-associated peptide epitope;
wherein said two polypeptide chains are fused to human IgG hinge domains and/or human IgG Fc domains or dimerizing portions thereof; and
wherein the said two polypeptide chains are connected by covalent and/or non- covalent bonds between said hinge domains and/or Fc-domains; and
wherein said dual specificity polypeptide molecule is capable of simultaneously binding the cell surface molecule and the MHC-associated peptide epitope, and dual specificity polypeptide molecules, wherein the order of the binding regions in the two polypeptide chains is selected from VD1 -VR1 and VR2-VD2 or VD1 - VR2 and VR1 -VD2, or VD2-VR1 and VR2-VD1 or VD2-VR2 and VR1 -VD1 and wherein the domains are either connected by LINK1 or LINK2, wherein the MHC-associated peptide epitope is VWGAOGVGK complex and the MHC is HLA-A*11.

Linkage of the specific binding molecule and the anti-CD3 antibody may be via covalent or noncovalent attachment. Covalent attachment may be direct, or indirect via a linker sequence. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. Examples of suitable linkers that may be used multi-domain binding molecules of the invention include, but are not limited to: GGGGS (SEQ ID No: 18), GGGSG (SEQ ID No: 19), GGSGG (SEQ ID No: 20), GSGGG (SEQ ID No: 21), GSGGGP (SEQ ID No: 22), GGEPS (SEQ ID No: 23), GGEGGGP (SEQ ID No: 24), and GGEGGGSEGGGS (SEQ ID No: 25) (as described in WO2010/133828) and GGGSGGGG (SEQ ID NO: 26). Additional linkers may include sequences having one or more of the following sequence motifs: GGGS (SEQ ID NO: 27), GGGGS (SEQ ID NO: 28), TVLRT (SEQ ID NO: 29), TVSSAS (SEQ ID NO: 30) and TVLSSAS (SEQ ID NO: 31).

Specific embodiments of anti-CD3-specific binding molecule fusion constructs of the invention include those alpha and beta chain pairings in which the alpha chain is composed of a TCR variable domain comprising the amino acid sequence of SEQ ID NOs: 4-6 and/or the beta chain is composed of a TCR variable domain comprising the amino acid sequence of SEQ ID NOs: 7-8. Said alpha and beta chains may further comprise a constant region comprising a non-native disulphide bond. The constant domain of the alpha chain may be truncated by eight amino acids. The N or C terminus of the alpha and or beta chain may be fused to an anti-CD3 scFv antibody fragment via a linker selected from SEQ ID NOs: 18-31. Certain preferred embodiments of such anti-CD3-specific binding molecule fusion constructs are provided in the table below and depicted in FIG. 3.

Specific binding molecules linked to anti-CD3

| | |
|---|---|
| Alpha chain | Beta Chain - antiCD3 |
| SEQ ID No: 9 | SEQ ID No: 10 |
| SEQ ID No: 11 | SEQ ID No: 12 |
| SEQ ID No: 13 | SEQ ID No: 14 |
| SEQ ID No: 15 | SEQ ID No: 16 |
| SEQ ID No: 17 | SEQ ID No: 18 |
| SEQ ID No: 19 | SEQ ID No: 20 |

A preferred specific binding molecule linked to antiCD3 comprises SEQ ID NO: 9 and SEQ ID NO: 10.

Also included within the scope of the invention are functional variants (also known as phenotypically silent variants) of said anti-CD3-TCR fusion constructs. Said functional variants preferably have at least 90% identity, such as 90%, 91%, 92%, 93%, 94%, 95%, 96%, 97%, 98%, 99% or 100% identity to the reference sequence, but are nonetheless functionally equivalent.

In a further aspect, the present invention provides nucleic acid encoding a specific binding molecule, or specific binding molecule anti-CD3 fusion of the invention. In some embodiments, the nucleic acid is cDNA. In some embodiments the nucleic acid may be mRNA, for example, mRNA encoded bispecific molecules (Stadler et al., Nat Med. 2017 Jul;23(7):815-817). In some embodiments, the invention provides nucleic acid comprising a sequence encoding an α chain variable domain of a TCR of the invention. In some embodiments, the invention provides nucleic acid comprising a sequence encoding a β chain variable domain of a specific binding molecule of the invention. The nucleic acid may be non-naturally occurring and/or purified and/or engineered. The nucleic acid sequence may be codon optimised, in accordance with expression system utilised. As is known to those skilled in the art, expression systems may include bacterial cells such as E. coli, or yeast cells, or mammalian cells, or insect cells, or they may be cell free expression systems. In some embodiment the molecules may be mRNA encoded bispecific antibodies.

In another aspect, the invention provides a vector which comprises nucleic acid of the invention. Preferably the vector is a TCR expression vector. Suitable TCR expression vectors include, for example, gamma-retroviral vectors or, more preferably, lentiviral vectors. Further details can be found in Zhang 2012 and references therein (Zhang et al,. Adv Drug Deliv Rev. 2012 Jun 1; 64(8): 756-762).

The invention also provides a cell harbouring a vector of the invention, preferably a TCR expression vector. Suitable cells include, mammalian cells, preferably immune cells, even more preferably T cells. The vector may comprise nucleic acid of the invention encoding in a single open reading frame, or two distinct open reading frames, encoding the alpha chain and the beta chain respectively. Another aspect provides a cell harbouring a first expression vector which comprises nucleic acid encoding the alpha chain of a specific binding molecule of the invention, and a second expression vector which comprises nucleic acid encoding the beta chain of a specific binding molecule of the invention. Such cells are particularly useful in adoptive therapy. The cells of the invention may be isolated and/or recombinant and/or non-naturally occurring and/or engineered.

Since the specific binding molecules of the invention have utility in adoptive therapy, the invention includes a non-naturally occurring and/or purified and/or or engineered cell, especially a T-cell, presenting a specific binding molecule of the invention. The invention also provides an expanded population of T cells presenting a specific binding molecule of the invention. There are a number of methods suitable for the transfection of T cells with nucleic acid (such as DNA, cDNA or RNA) encoding the specific binding molecules of the invention (see for example Robbins et al., (2008) J Immunol. 180: 6116-6131). T cells expressing the specific binding molecules of the invention will be suitable for use in adoptive therapy-based treatment of cancer. As will be known to those skilled in the art, there are a number of suitable methods by which adoptive therapy can be carried out (see for example Rosenberg et al., (2008) Nat Rev Cancer 8(4)).

As is well-known in the art, in vivo production of proteins including those comprising the specific binding molecules of the invention may result in post translational modifications. Glycosylation is one such modification, which comprises the covalent attachment of oligosaccharide moieties to defined amino acids in the polypeptide chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody based therapeutics. (Jefferis et al., (2009) Nat Rev Drug Discov

Mar;8:226-34.). For the specific binding molecules of the invention glycosylation may be controlled, by using particular cell lines for example (including but not limited to mammalian cell lines such as Chinese hamster ovary (CHO) cells or human embryonic kidney (HEK) cells), or by chemical modification. Such modifications may be desirable, since glycosylation can improve pharmacokinetics, reduce immunogenicity and more closely mimic a native human protein (Sinclair and Elliott, (2005) Pharm Sci.Aug; 94(8):1626-35). In some cases, mutations may be introduced to control and or modify post translational modifications.

For administration to patients, the specific binding molecules of the invention (preferably associated with a detectable label or therapeutic agent or expressed on a transfected T cell), specific binding molecule-anti CD3 fusion molecules, nucleic acids, expression vectors or cells of the invention may be provided as part of a sterile pharmaceutical composition together with one or more pharmaceutically acceptable carriers or excipients. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, such as parenteral (including subcutaneous, intramuscular, intrathecal or intravenous), enteral (including oral or rectal), inhalation or intranasal routes. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated, the age and condition of the individual to be treated, etc. a suitable dose range for a specific binding molecule-anti-CD3 fusion molecules may be in the range of 25 ng/kg to 50 µg/kg or 1 µg to 1 g. A physician will ultimately determine appropriate dosages to be used. An example of a suitable dosing regimen is provided in WO2017208018.

Specific binding molecules, specific binding molecule-anti-CD3 fusion molecules, pharmaceutical compositions, vectors, nucleic acids and cells of the invention may be provided in substantially pure form, for example, at least 80%, at least 85%, at least 90%, at least 91%, at least 92%, at least 93%, at least 94%, at least 95%, at least 96%, at least 97%, at least 98%, at least 99% or 100% pure.

Also provided by the invention are:
A specific binding molecule, specific binding molecule-anti-CD3 fusion molecule, nucleic acid, pharmaceutical composition or cell of the invention for use in medicine, preferably for use in a method of treating cancer, including but not limited to pancreatic, colorectal, lung (including non-small cell lung cancer), ovarian (including clear cell, endometrioid, mucinous) gastrointestinal (including bile duct, gall bladder, small intestine, ampulla, ceacum, appendix) and endometrial. Particularly preferred cancer indications are pancreatic and colorectal.
the use of a specific binding molecule, specific binding molecule-anti-CD3 fusion molecule, nucleic acid, pharmaceutical composition or cell of the invention in the manufacture of a medicament for treating cancer, including but not limited to pancreatic, colorectal, lung (including non-small cell lung cancer), ovarian (including clear cell, endometrioid, mucinous) gastrointestinal (including bile duct, gall bladder, small intestine, ampulla, ceacum, appendix) and endometrial. Particularly preferred cancer indications are pancreatic and colorectal.
a method of treating cancer, comprising administering to a subject in need thereof a therapeutically effective amount of a specific binding molecule-anti-CD3 fusion molecule, including but not limited to pancreatic, colorectal, lung (including non-small cell lung cancer), ovarian (including clear cell, endometrioid, mucinous) gastrointestinal (including bile duct, gall bladder, small intestine, ampulla, ceacum, appendix) and endometrial. Particularly preferred cancer indications are pancreatic and colorectal.
an injectable formulation for administering to a human subject comprising a specific binding molecule, specific binding molecule-anti-CD3 fusion molecule, nucleic acid, pharmaceutical composition or cell of the invention.

The specific binding molecule, specific binding molecule-anti-CD3 fusion molecule, nucleic acid, pharmaceutical composition or cell of the invention may be administered by injection, such as intravenous, subcutaneous, or direct intratumoral injection. The human subject may be of the HLA-A*02 subtype. The patient may undergo screening prior to treatment to determine expression of the mutant Kras protein and or the presence of the mutant peptide. Additionally or alternatively, the patient may be screened for HLA-A11. Where treatment of a tumour is contemplated, the tumour may be a solid or a liquid tumour.

The method of treatment may further include administering separately, in combination, or sequentially, one or more additional anti-neoplastic agents.

The terms “treatment,” “treat,” “treating,” and the like, are meant to include slowing, stopping, or reversing the progression of cancer. These terms also include alleviating, ameliorating, attenuating, eliminating, or reducing one or more symptoms of a disorder or condition, even if the cancer is not actually eliminated and even if progression of the cancer is not itself slowed, stopped or reversed.

“Therapeutically effective amount” means the amount of a compound, or pharmaceutically acceptable salt thereof, administered to the subject that will elicit the biological or medical response of or desired therapeutic effect on a subject.

A therapeutically effective amount can be readily determined by the attending clinician, as one skilled in the art, by the use of known techniques and by observing results obtained under analogous circumstances. In determining the effective amount for a subject, a number of factors are considered by the attending clinician, including, but not limited to: size, age, and general health; the specific disease or disorder involved; the degree of or involvement or the severity of the disease or disorder; the response of the individual subject; the particular compound administered; the mode of administration; the bioavailability characteristics of the preparation administered; the dose regimen selected; the use of concomitant medication; and other relevant circumstances.

Preferred features of each aspect of the invention are as for each of the other aspects mutatis mutandis. The prior art documents mentioned herein are incorporated by reference to the fullest extent permitted by law.

### DESCRIPTION OF THE DRAWINGS

FIG. 1 provides amino acid sequences of alpha and beta variable and constant domains of a soluble scaffold TCR. The CDR sequences are underlined.

FIG. 2 provides example amino acid sequences of mutated TCR alpha and beta variable domain. The CDRs are underlined and mutations relative to the WT sequence are shown in bold.

FIG. 3 provides example amino acid sequences of TCR-antiCD3 fusion proteins incorporating mutated TCR variable domains.

FIG. 4 provides exemplary graphical data demonstrating TCR-antiCD3 fusion proteins are able to drive potent T cell activation in the presence of cells pulsed with the mutant KRAS peptide (labelled VVV(D) K-RAS^{G12D}), relative to cells pulsed with the WT KRAS peptide (labelled VVV(G) wt K-RAS). IFNy release is used as a readout for T cell activation.

FIG. 5 provides exemplary graphical data demonstrating TCR-antiCD3 fusions proteins are able to drive potent T cell activation in the presence of cancer cell lines that express mutant KRAS (Panc-1×A11β2M and CL40). Cell lines NCI-H2030 and SK-Mel-28 express WT KRAS. IFNy release is used as a readout for T cell activation.

FIG. 6 provides exemplary graphical data demonstrating TCR-antiCD3 fusions mediate potent killing of a cancer cell line that expresses the mutant KRAS peptide (CL40), relative to a cell line that expresses the WT KRAS peptide (SK-Mel-28). The percentage of target cells remaining at 72 h is used as a marker of target cell death.

FIG. 7 provides exemplary graphical data demonstrating TCR-antiCD3 fusion proteins result in little or no activity against cell lines derived from normal tissues (Normal cells) at concentrations below 1 nM. Panc-1×A11β2M and SK-Mel-28 cell are positive and negative controls respectively. IFNy release is used as a readout for T cell activation.

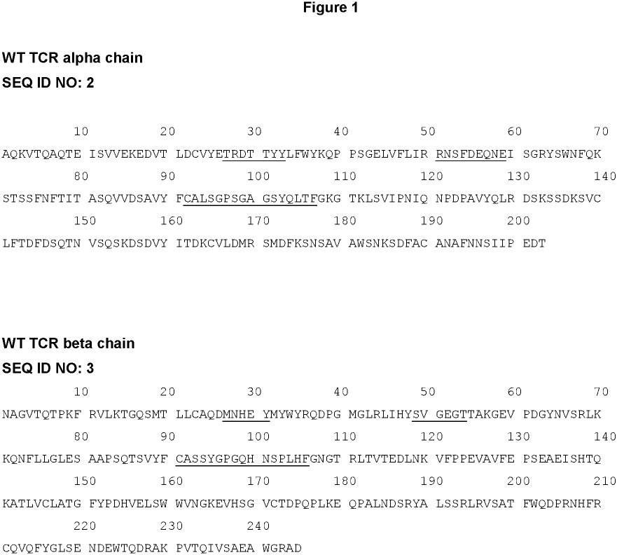

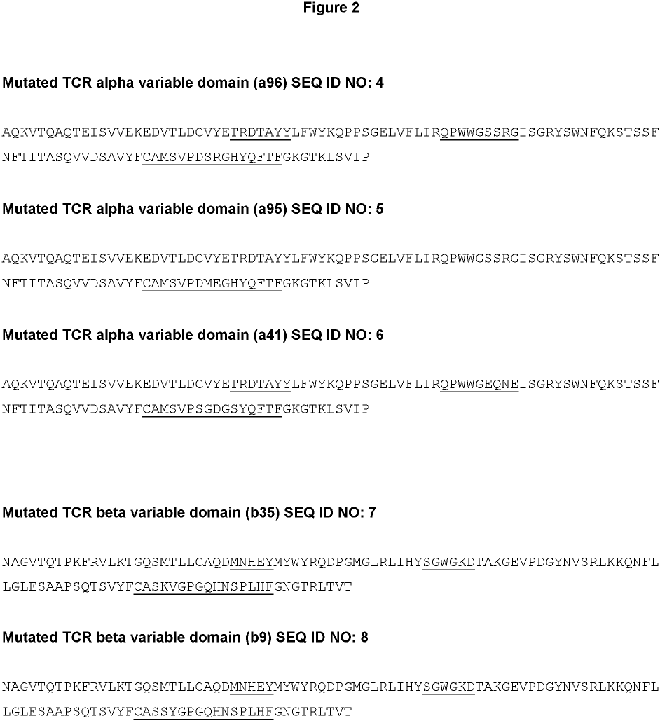

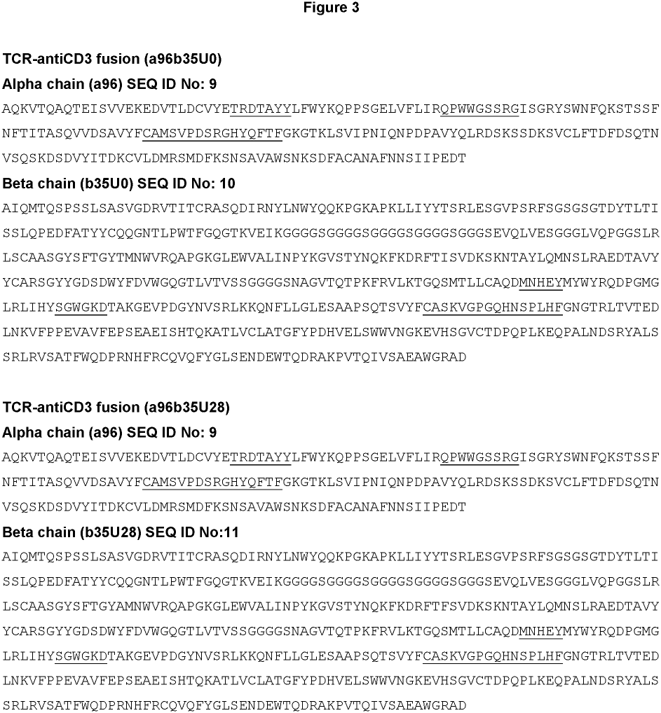

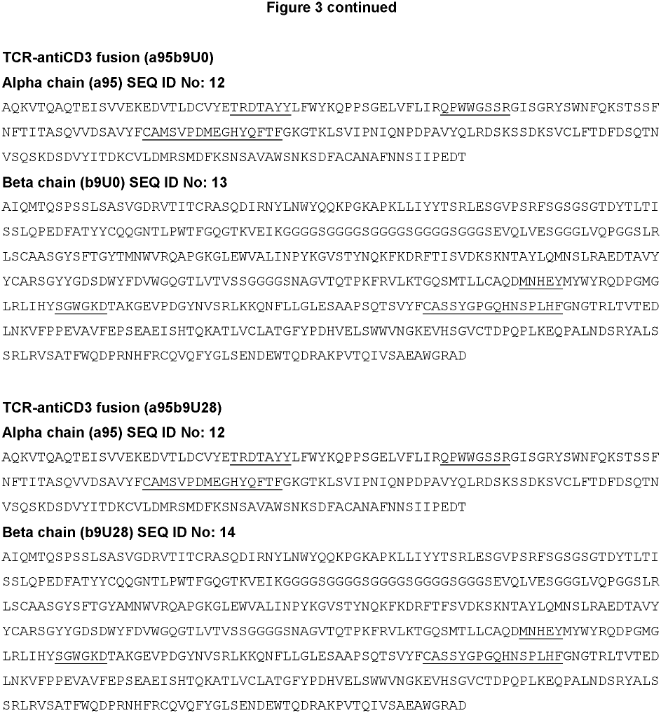

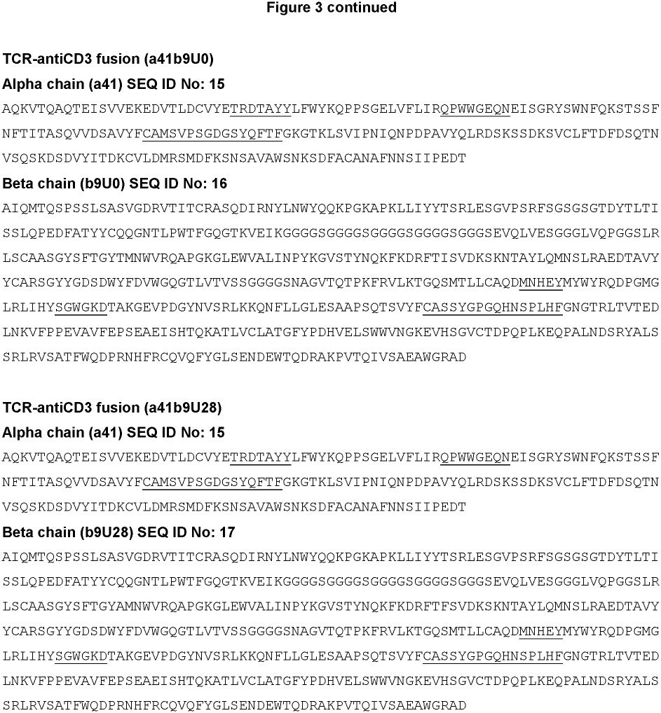

The invention is further described in the following non-limiting examples.

### EXAMPLES

### Example 1 - Isolation and Characterisation of WT TCR

### A) Preparation of Soluble WT TCR

A TCR that recognises the VVVGADGVGK-HLA-A*11 complex was identified from donor PBMCs using known T cell cloning methodology, and TCR chains subsequently identified by RACE. The WT TCR was prepared as a soluble alpha beta heterodimer as previously described (Boulter et al., Protein Eng. 2003 Sep;16(9):707-11 and WO03/020763).

Briefly DNA sequences encoding the alpha and beta extracellular regions of a soluble TCR comprising the amino acid sequences provided in SEQ ID Nos 1 and 2 were cloned separately into an expression plasmid using standard methods and transformed separately into E. coli strain Rosetta 2(DE3)pLysS. For expression, cells were grown in auto-induction media supplemented with 1% glycerol (+ 100 µg/ml ampicillin and 34 µg/ml chloramphenicol) for 2 hours at 37° C. before reducing the temperature to 30° C. and incubating overnight. Harvested cell pellets were lysed with BugBuster protein extraction reagent (Merck Millipore). Inclusion body pellets were recovered by centrifugation, washed twice in Triton buffer (50 mM Tris-HCI pH 8.1, 0.5% Triton-X100, 100 mM NaCl, 10 mM NaEDTA) and finally resuspended in detergent free buffer (50 mM Tris-HCl pH 8.1, 100 mM NaCl, 10 mM NaEDTA).

For refolding, inclusion bodies were first mixed and diluted into solubilisation/denaturation buffer (6 M Guanidine-hydrochloride, 50 mM Tris HCl pH 8.1, 100 mM NaCl, 10 mM EDTA, 20 mM DTT) followed by incubation for 30 min at 37° C. Refolding is then initiated by further dilution into refold buffer (100 mM Tris pH 8.1, 800 or 400 mM L-Arginine HCI, 2 mM EDTA, 4 M Urea, 6.5 mM cysteamine hydrochloride and 1.9 mM cystamine dihydrochloride). The refolded mixture was then dialysed against 10 L H₂O per L of refold for 18-20 hours at 5° C. ± 3° C. After this time, the dialysis buffer was twice replaced with 10 mM Tris pH 8.1 (10 L) and dialysis continued for a further 15 hours. The dialysed mixture was then filtered through 0.45 µm cellulose filters. The sample was then applied to a POROS® 50HQ anion exchange column and bound protein eluted with a gradient of 0-500 mM NaCl in 20 mM Tris pH 8.1, over 6 column volumes. Peak fractions were identified by SDS PAGE before being pooled and concentrated. The concentrated sample was then applied to a Superdex® 200 Increase 10/300 GL gel filtration column (GE Healthcare) pre-equilibrated in Dulbecco’s PBS buffer. The peak fractions were pooled and concentrated.

### B) Biophysical Characterisation of the Soluble WT TCR

Binding of the soluble TCR to the VVVGADGVGK-HLA-A*11 complex was assessed using surface plasmon resonance (SPR). Binding specificity was determined by measuring cross recognition of the non-mutated KRAS peptide VVVGAGGVGK, as well as additional peptides with high sequence homology and / or having the same binding motif as identified by an alanine scanning approach. Cross reactively to a further pool of commonly presented HLA-A11 peptides of various lengths peptides was also assessed (termed CPmix).

First, truncated and biotinylated HLA-A11 heavy chain and human beta 2-microglobulin (β2m) were prepared from E. coli as inclusion bodies and refolded and purified as previously described (Garboczi, Hung, & Wiley, 1992; O’Callaghan et al., 1999). Biotinylated peptide-HLA monomers were subsequently immobilized onto streptavidin-coupled CM-5 Series S sensor chips. Equilibrium binding constants were determined using serial dilutions of the soluble TCR injected at a constant flow rate of 10-30µl min-1 over a flow cell coated with approximately 500 response units (RU) of peptide-HLA complex. Equilibrium responses were normalised for each TCR concentration by subtracting the bulk buffer response on a control flow cell containing no peptide-HLA. The K_{D} value is obtained by non-linear curve fitting using GraphPad Prism 8 software and the Langmuir binding isotherm; bound = C*Max/(C + KD), where “bound” is the equilibrium binding in RU at injected TCR concentration C and Max is the maximum binding. All measurements are performed at 25° C. in Dulbecco’s PBS buffer, supplemented with 0.005% surfactant P20.

### Results

The binding properties for the interaction between the soluble WT TCR and various peptide-HLA-A11 complexes are shown below

| | | |
|---|---|---|
| Protein | peptide | Affinity K_{D} (µM) |
| KRAS G12D (10-mer) | VVVGADGVGK | 70 |
| KRAS G12D (9-mer) | VVGADGVGK | NB |
| KRAS WT | VVVGAGGVGK | NB |
| KRAS G12C | VVVGACGVGK | NB |
| ERAS | VVVGASGVGK | NB |
| RRAS | VVVGGGGVGK | NB |
| MRAS | VVVGDGGVGK | NB |
| DIRAS2 | VVFGAGGVGK | NB |
| RAP1A | VVLGSGGVGK | NB |
| A11-10-CPmix01 | 10 decamer peptides | NB |
| A11-10-CPmix02 | 10 decamer peptides | NB |
| A11-9-CPmix01 | 8 nonamer peptides | NB |

NB = no binding

These data show that the WT TCR is able to specifically bind to the VVVGADGVGK-HLA-A*11 complex and is able to discriminate between the mutated and non-mutated KRAS peptide. In addition, no binding was detected to a number of additional peptides, including those with a high level of sequence homology.

### Example 2 - Generation of High Affinity TCRs and TCR-AntiCD3 Fusion Proteins

The soluble WT TCR described in Example 1 was used as a template to identify mutations that increase the binding affinity of the TCR for peptide HLA complex, using phage display and random mutagenesis techniques known in the art (for example see Li et al., Nat Biotechnol. 2005 Mar;23(3):349-54). The non-mutated KRAS peptide was used for deselection during the phage display process. High affinity TCRs were subsequently prepared as bispecific fusion proteins comprising a soluble TCR fused to an anti-CD3 scFV.

### A) Preparation of Soluble TCR-AntiCD3 Fusion Proteins

The same process was followed as described for soluble TCRs in Example 1, except that the TCR beta chain was fused via a linker to an anti-CD3 single chain antibody. In addition, the concentration of the redox reagents in the refolding step was 1 mM cystamine dihydrochloride, 10 mM cysteamine hydrochloride). Finally, a cation exchange step was added following the anion exchange step. In this case, the peak fractions from anion exchange were diluted 20-fold in 40 mM MES pH 6.2 and applied to a POROS® 50HS cation exchange column. Bound protein was eluted with a gradient of 0-500 mM NaCl in 40 mM MES. Peak fractions were pooled and adjusted to 200 mM Tris pH 8.1, before being concentrated and applied directly to the gel filtration matrix.

### B) Biophysical Characterisation of Soluble TCR-AntiCD3 Fusion Proteins

Binding analysis was carried out using similar SPR methodology as described in Example 1. Except that for high affinity interactions, binding parameters were determined by single cycle kinetics analysis. Five different concentrations of soluble TCR or fusion protein were injected over a flow cell coated with ~50 - 200 RU of peptide-HLA complex using a flow rate of 50-60 µl min⁻¹. Typically, 60-200 µl of soluble TCR or fusion molecule was injected at a top concentration of between 2-100 nM, with successive 2 fold dilutions used for the other four injections. The lowest concentration was injected first. To measure the dissociation phase, buffer was injected until ≥ 10% dissociation occurred, typically after 1 - 3 hours. Kinetic parameters were calculated using the manufacturer’s software. The dissociation phase was fitted to a single exponential decay equation enabling calculation of half-life. The equilibrium constant K_{D} was calculated from k_{off}/kₒₙ.

| TCR-CD3 fusion | K_{D} (pM) | kₒₙ (ka) (⅟Ms) | k_{off} (kd) | t_{½} (h) | Affinity window K_{D} WT/K_{D} G12D |
|---|---|---|---|---|---|
| a96b35U28 | 66 | 1.55E+05 | 1.02E-05 | 19 | >3000 |
| a96b35U | 46 | 2.15E+05 | 9.93E-06 | 19 | >3000 |
| a95b9U28 | 87 | 1.11E+05 | 9.63E-06 | 20 | >3000 |
| a95b9U | 70 | 1.39E+05 | 9.77E-06 | 20 | >3000 |
| a41b9U28 | 172 | 5.61E+04 | 9.63E-06 | 20 | >3000 |
| a41b9U | 168 | 5.72E+04 | 9.63E-06 | 20 | >3000 |

These data show that the high affinity variants retain binding specificity to the VVVGADGVGK-HLA-A*11 complex and are able to discriminate between mutated and non-mutated KRAS peptide.

### Example 3 - Cellular Analysis of Soluble TCR-AntiCD3 Fusion Proteins

Soluble TCR-antiCD3 fusion proteins mediate potent and specific T cell activation

### A) Peptide Pulsed Cells

TCR-antiCD3 fusions proteins were tested for their ability to mediate T cell activation in the presence of target cells pulsed with either mutant G12D peptide or the WT peptide.

T cell activation was assessed using IFNγ release and detected using an ELISPOT assay kit. HLA-A11+ve SUP-B15 cells were used as target cells and pulsed with 10 µM of peptide. HLA-A11+ PBMCs obtained from donor blood were used as effector cells. The effector to target ratio was 1:1. Assays were performed using a human IFN-γ ELISPOT kit (BD Biosciences) according to the manufacturer’s instructions. Briefly, ELISPOT plates were coated with IFNy antibody 1-7 days before assay. On the day of the assay, ELISPOT plates were blocked with 100 µl assay medium (R10). After removal of block, target cells were plated at 50,000/well in 50 µl. Fusion protein were titrated to give final concentrations spanning the anticipated biologically active range (typically a top concentration of 10 nM with log or semi-log dilutions), and added to the well in a volume of 50 µl. Effector cells were thawed from liquid nitrogen counted and plated at 40-50,000 cells/well in 50 µl (the exact number of cells used for each experiment is donor dependent and may be adjusted to produce a response within a suitable range for the assay). The final volume of each well was made up to 200 µl with R10. The plates/cells were cultured overnight and the next day the plates were washed, assayed following the manufacturer’s instructions and allowed to dry at room temperature for at least 2 hours prior to counting the spots using a CTL analyser with Immunospot software (Cellular Technology Limited). Dose response curves were plotted using PRISM software.

Controls included samples prepared with i) targets and or effectors alone, ii) effectors and 10 nM TCR-antiCD3 fusion.

### Results

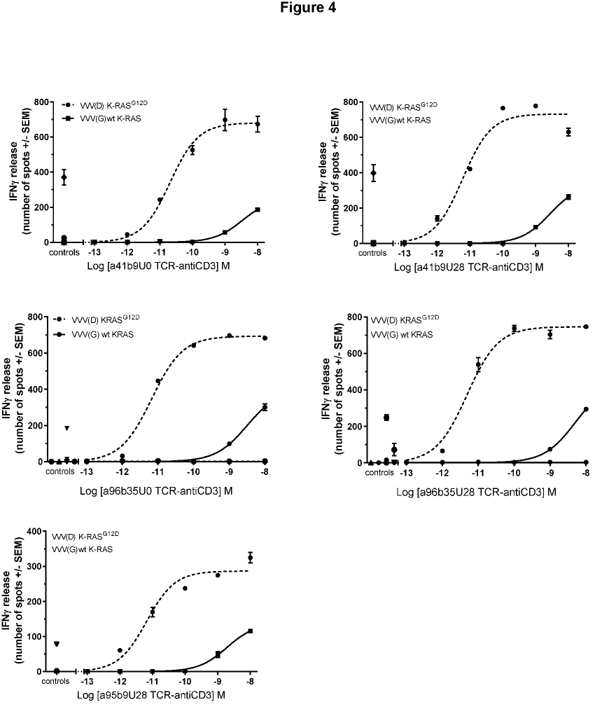

The TCR-antiCD3 fusion proteins of the invention resulted in potent and specific T cell activation in the presence of cells presenting the mutant Kras peptide (VVVGADGVGK) HLA-A*11 complex. In each case there was at least a 100 fold difference in the concentration required for T cell activation between the mutant and WT peptides, indicating that the TCR-antiCD3 fusion proteins can sufficiently discriminate between the mutant and WT peptide. Graphical data for 5 TCR-antiCD3 fusions proteins are provided in FIG. 4.

### B) Cell Lines

T cell activation by TCR-antiCD3 fusion proteins was further tested using cell lines that are either positive or negative for antigen.

In this example, the following human cancer cells lines were used as target cells:
Panc-1×A11β2M (Pancreas) antigen positive (KRAS G12D positive; transduced with HLA-A11 and β2M)
CL40 (Colorectal) antigen positive (KRAS G12D positive)
SK-Mel-28 (Melanoma) antigen negative (wt KRAS positive)
NCl-H2030 (Lung) antigen negative (KRAS G12C positive)

Cell lines were treated with a 6-point concentration range of TCR-antiCD3 fusion protein and co-cultured with HLA-A11+ PBMCs obtained from donor blood at an effector to target ratio of 0.8:1. IFNy release was measured by ELISPOT assay as described above.

### Results

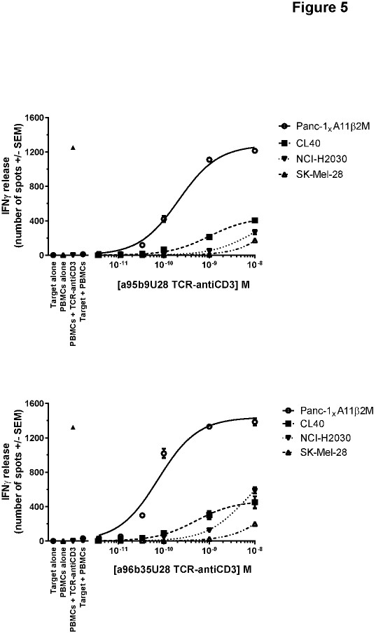

The TCR-antiCD3 fusion proteins of the invention mediate potent T cell activation in the presence of cells that naturally present the mutant KRAS peptide, with EC₅₀ values in the picomolar range (≤ 1000 pM). Cell lines that present the WT peptide, or an alternative mutant peptide, resulted in little or no T cell activation at concentrations of TCR-antiCD3 fusion below 1 nM. Graphical data for two TCR-antiCD3 fusions proteins are presented in FIG. 5

### C) Soluble TCR-AntiCD3 Fusion Proteins Mediate Potent and Specific Killing of Cancer Cell Lines TCR-AntiCD3 Fusions Proteins Were Tested for Their Ability to Drive T Cell Mediated Killing of Cancer Cell Lines That are Either Positive or Negative for Antigen

In this example CL40 and SK-Mel-28 were used as positive and negative target cells respectively. Target cells were treated with a 7 point concentration range of TCR-antiCD3 fusion proteins and co-cultured with HLA-A11+ PBMC in the presence of a caspase sensitive green fluorescent probe for 72 h using the IncuCyte ZOOM platform. Images were acquired every 2 h and redirected T cell killing of red fluorescent target cells was detected and analysed using the Incucyte ZOOM software. Dose response curves were plotted and IC₅₀ values calculated using PRISM software.

### Results

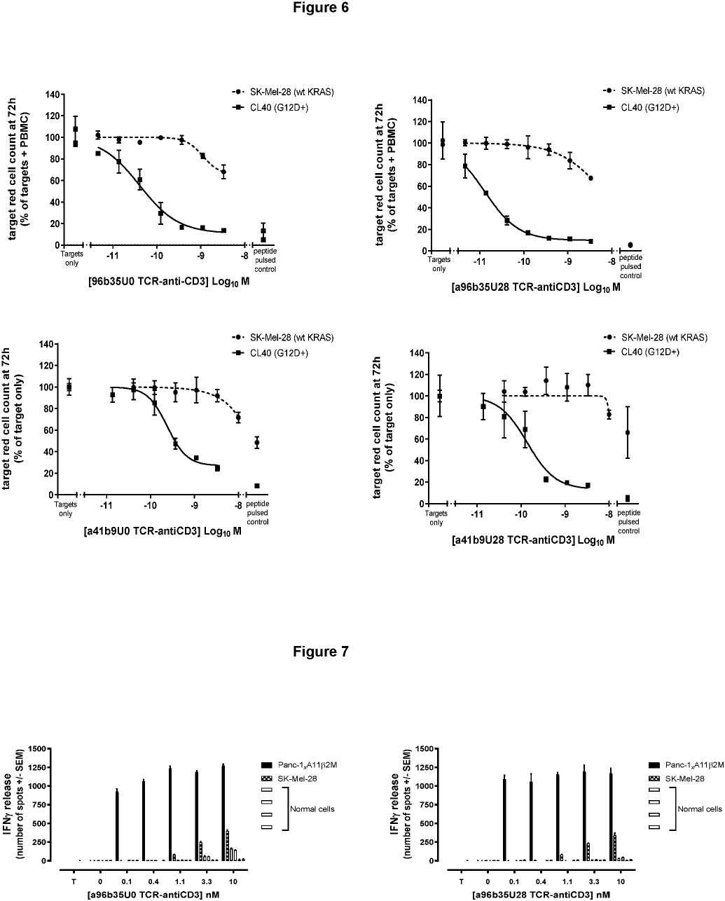

IC50 value for each of the TCR-antiCD3 fusion proteins in the presence of antigen positive cells are shown in the table below. Graphical data for four TCR-antiCD3 fusions proteins are presented in FIG. 6

| TCR-antiCD3 fusion | IC50 (pM) | R² |
|---|---|---|
| a95b9 | 122.6 | 0.8993 |
| a95b9U28 | 120.6 | 0.8115 |
| a96b35 | 112.1 | 0.7497 |
| a96b35U28 | 56.6 | 0.9717 |
| a41b9 | 240 | 0.9571 |
| a41b9U28 | 137.4 | 0.8942 |

These data show that the TCR-antiCD3 fusion proteins of the invention drive potent T cell mediated killing of a colorectal cancer cell line that naturally presents the VVVGADGVGK-HLA-A*11 complex. IC50 values are in the picomolar range (≤ 1000 pM). Little or no T cell mediated killing of SK-Mel-28 cells was observed at concentrations of TCR-antiCD3 fusion below 1 nM.

### Example 4 - Further Specificity Testing of TCR Anti-CD3 Fusions Proteins

TCR-antiCD3 fusions proteins were further tested for suitability as therapeutic reagents by assessing T cell activation in the presence of a panel of cell lines derived from normal healthy tissues.

Cell lines were treated with a 6-point concentration range of TCR-antiCD3 fusion protein and co-cultured with HLA-A11+ PBMCs obtained from donor blood at an effector to target ratio of 1:1. IFNy release was measured by ELISPOT assay as described above. Panc-1×A11 and SK-Mel-28 were used as positive and negative controls respectively.

### Results

These data indicate the TCR-antiCD3 fusion proteins of the invention give rise to minimal,or no, T cell activity against various normal tissues at concentrations of ≤1 nM. Graphical data for two TCR-antiCD3 fusions proteins are presented in FIG. 7

1. A specific binding molecule having the property of binding to VVVGADGVGK (SEQ ID NO: 1) HLA-A*11 complex and comprising a TCR alpha chain variable domain and/or a TCR beta chain variable domain each of which comprises FR1-CDR1-FR2-CDR2-FR3-CDR3-FR4 where FR is a framework region and CDR is a complementarity determining region, wherein
(a) the alpha chain CDRs have the following sequences:
CDR1 — TRDTTYY (SEQ ID No: 32),
CDR2 — RNSFDEQNE (SEQ ID No: 33),
CDR3 — CALSGPSGAGSYQLTF (SEQ ID No: 34),
optionally with one or more mutations therein, and/or
(b) the beta chain CDRs have the following sequences:
CDR1 — MNHEY (SEQ ID No: 35),
CDR2 — SVGEGT (SEQ ID No: 36),
CDR3 — CASSYGPGQHNSPLHF (SEQ ID No: 37),
optionally with one or more mutations therein.

2. The specific binding molecule of claim 1, wherein the alpha chain variable domain framework regions comprise the following sequences:
FR1 — amino acids 1-26 of SEQ ID NO: 2,
FR2 — amino acids 34-50 of SEQ ID NO: 2,
FR3 — amino acids 60-91 of SEQ ID NO: 2,
FR4 - amino acids 108-117 of SEQ ID NO: 2,
or respective sequences having at least 90, 91, 92, 93, 94, 95, 96, 97, 98 or 99% identity to said sequences, and/or
the beta chain variable domain framework regions comprise the following sequences:
FR1 — amino acids 1-26 of SEQ ID NO: 3,
FR2 — amino acids 32-48 of SEQ ID NO: 3,
FR3 — amino acids 55-90 of SEQ ID NO: 3,
FR4 — amino acids 106-115 of SEQ ID NO: 3,
or respective sequences having at least 90, 91, 92, 93, 94, 95, 96, 97, 98 or 99% identity to said sequences.

3. The specific binding molecule of any preceding claim, wherein one or more of the mutations in the alpha chain CDRs is selected from: T31A, R51Q, N52P, S53W, F54W, D55G, E56S, Q57S, N58R, E59G, L94M, G96V, S98D, G99S or G99M, A100R or A100E or A100D, S102H, L105F, with reference to the numbering of SEQ ID NO: 2 and/or
one or more of the mutations in the beta chain CDRs is selected from: V50G, G51W, E52G, G53K, T54D, S94K, Y95V, with reference to the numbering of SEQ ID NO: 3.

4. The specific binding molecule of any preceding claim, wherein the alpha chain CDR1, CDR2 and CDR3 sequences are selected from:

| | |
|---|---|
| CDR1 | TRDTTYY (SEQ ID No: 32), or |
| | TRDTAYY (SEQ ID No: 38), |
| CDR2 | RNSFDEQNE (SEQ ID No: 33), |
| | QPWWGSSRG (SEQ ID No: 39), or |
| | QPWWGEQNE (SEQ ID No: 40), |
| CDR3 | CALSGPSGAGSYQLTF(SEQ ID No: 34), |
| | CAMSVPDSRGHYQFTF (SEQ ID No: 41), |
| | CAMSVPDMEGHYQFTF (SEQ ID No: 42), or |
| | CAMSVPSGDGSYQFTF (SEQ ID No: 43), |

and/or
the beta chain CDR1, CDR2 and CDR3 sequences are selected from

| | |
|---|---|
| CDR1 | MNHEY (SEQ ID No: 35), |
| CDR2 | SVGEGT (SEQ ID No: 36), or |
| | SGWGKD (SEQ ID No: 44), |
| CDR3 | CASSYGPGQHNSPLHF (SEQ ID No: 45), or |
| | CASKVGPGQHNSPLHF (SEQ ID No: 46). |

.

5. The specific binding molecule of any preceding claim, wherein
in the alpha chain CDR1 is TRDTAYY, CDR2 is QPWWGSSRG and CDR3 is CAMSVPDSRGHYQFTF, and in the beta chain CDR1 is MNHEY, CDR2 is SGWGKD and CDR3 is CASKVGPGQHNSPLHF or
in the alpha chain CDR1 is TRDTAYY, CDR2 is QPWWGSSRG and CDR3 is CAMSVPDMEGHYQFTF, and in the beta chain CDR1 is MNHEY, CDR2 is SGWGKD and CDR3 is CASSYGPGQHNSPLHF or
in the alpha chain CDR1 is TRDTAYY CDR2 is QPWWGEQNE and CDR3 is CAMSVPSGDGSYQFTF and in the beta chain CDR1 is MNHEY, CDR2 is SGWGKD and CDR3 is CASSYGPGQHNSPLHF.

6. A specific binding molecule as claimed in any preceding claim, wherein the alpha chain variable domain comprises any one of the amino acid sequences of SEQ ID NOs: 4-6 and the beta chain variable domain comprises any one of the amino acid sequences of SEQ ID NOs: 7-8.

7. A specific binding molecule claimed in any preceding claim wherein the alpha chain variable domain and the beta chain variable domain are selected from the amino acid sequences of:

| | |
|---|---|
| Alpha chain variable domain | Beta chain variable domain |
| SEQ ID No 4 | SEQ ID No 7 |
| SEQ ID No 5 | SEQ ID No 8 |
| SEQ ID No 6 | SEQ ID No 8 |

.

8. A specific binding molecule as claimed in any preceding claim, which is an alpha-beta heterodimer, having an alpha chain TRAC constant domain sequence and a beta chain TRBC1 or TRBC2 constant domain sequence.

9. A specific binding molecule as claimed in claim 8, wherein a non-native covalent disulphide bond links a residue of the constant domain of the alpha chain to a residue of the constant domain of the beta chain.

10. A specific binding molecule as claimed in any one of claims 1 to 8, which is in single chain format of the type Vα,-L-Vβ, Vβ-L-Vα, Vα-Cα-L-Vβ, Vα-L-Vβ-Cβ, wherein Vα and Vβ are TCR α and β variable regions respectively, Cα and Cβ are TCR α and β constant regions respectively, and L is a linker sequence.

11. A specific binding molecule as claimed in any one of claims 1-8, comprising
a first polypeptide chain which comprises the alpha chain variable domain and a first binding region of a variable domain of an antibody; and
a second polypeptide chain which comprises the beta chain variable domain and a second binding region of a variable domain of said antibody,
wherein the respective polypeptide chains associate such that the specific binding molecule is capable of simultaneously binding VVVGADGVGK (SEQ ID NO: 1) HLA-A*11 complex and an antigen of the antibody.

12. A specific binding molecule as claimed in any preceding claim associated with a detectable label, and/or a therapeutic agent, and/or a PK modifying moiety.

13. A specific binding molecule as claimed in claim 12, wherein an anti-CD3 antibody is covalently linked to the C- or N-terminus of the alpha or beta chain of the TCR, optionally via a linker sequence.

14. A specific binding molecule-anti-CD3 fusion molecule wherein the alpha chain variable domain comprises an amino acid sequence selected from SEQ ID NOs: 4-6 and the beta chain variable domain comprises an amino acid sequence selected from SEQ ID NO: 7-8, and wherein the anti-CD3 antibody is covalently linked to the N-terminus or C-terminus of the TCR beta chain via a linker sequence selected from SEQ ID NOs: 18-31.

15. A specific binding molecule-anti-CD3 fusion molecule as claimed in claim 14, comprising
an alpha chain amino acid sequence as set forth in SEQ ID NO: 9, or 12, or 15, or an alpha chain amino acid sequence that has at least 90% identity, such as at least 90%, at least 91%, at least 92%, at least 93%, at least 94%, at least 95%, at least 96%, at least 97%, at least 98%, at least 99% or 100% identity, to the amino acid sequences as set forth in SEQ ID NO: 9, or 12, or 15,
and a beta chain amino acid sequence as set forth inSEQ ID NO: 10, or 11, or 13, or 14, or 16, or 17, or a beta chain amino acid sequence that has at least 90% identity, such as at least 90%, at least 91%, at least 92%, at least 93%, at least 94%, at least 95%, at least 96%, at least 97%, at least 98%, at least 99% or 100% identity, to the amino acid sequences as set forth in SEQ ID No: 10, or 11, or 13, or 14, or 16, or 17.

16. A specific binding molecule-anti CD3 fusion molecule as claimed in claim 15, comprising
(a) an alpha chain amino acid sequence corresponding to SEQ ID NO: 9 and beta chain amino acid sequence corresponding to SEQ ID NO: 10;
(b) an alpha chain amino acid sequence corresponding to SEQ ID NO: 9 and beta chain amino acid sequence corresponding to SEQ ID NO: 11; or
(c) an alpha chain amino acid sequence corresponding to SEQ ID NO: 12 and beta chain amino acid sequence corresponding to SEQ ID NO: 13.
(d) an alpha chain amino acid sequence corresponding to SEQ ID NO: 12 and beta chain amino acid sequence corresponding to SEQ ID NO: 14.
(e) an alpha chain amino acid sequence corresponding to SEQ ID NO: 15 and beta chain amino acid sequence corresponding to SEQ ID NO: 16.
(f) an alpha chain amino acid sequence corresponding to SEQ ID NO: 15 and beta chain amino acid sequence corresponding to SEQ ID NO: 17.

17. A nucleic acid encoding a TCR alpha chain and/or a TCR beta chain as claimed in any one of the preceding claims.

18. An expression vector comprising the nucleic acid of claim 17.

19. A cell harbouring
(a) an expression vector as claimed in claim 18 encoding TCR alpha and beta variable chains as claimed in any one of claims 1 to 16, in a single open reading frame, or two distinct open reading frames; or
(b) a first expression vector which comprises nucleic acid encoding the alpha variable chain of a TCR as claimed in any one of claims 1 to 16, and a second expression vector which comprises nucleic acid encoding the beta variable chain of a TCR as claimed in any one of claims 1 to 16.

20. A non-naturally occurring and/or purified and/or engineered cell, especially a T-cell, presenting a specific binding molecule as claimed in any one of claims 1 to 16.

21. A pharmaceutical composition comprising a specific binding molecule as claimed in any one of claims 1-13, or a specific binding molecule-anti CD3 fusion molecule as claimed in any one of claims 14-16, a nucleic acid as claimed in claim 17, an expression vector as claimed in claim 18, and/or a cell as claimed in claim 19 or 20, together with one or more pharmaceutically acceptable carriers or excipients.

22. The specific binding molecule of any one of claims 1 to 13, specific binding molecule -anti-CD3 fusion molecule of any one of claims 14-16, nucleic acid of claim 17, e, cell of claim 19 or 20 and/or pharmaceutical composition of claim 21, for use in medicine, preferably in a human subject.

23. The specific binding molecule of any one of claims 1 to 13, or specific binding molecule -anti-CD3 fusion molecule of any one of claims 14-16, nucleic acid of claim 17, expression vector of claim 18, cell of claim 19 or 20 and/or pharmaceutical composition of claim 21, for use in a method of treating cancer, preferably in a human subject.

24. A method of producing a specific binding molecule according to any one of claims 1 to 13, or a specific binding molecule-anti-CD3 fusion molecule according to any one of claims 14-16, comprising a) maintaining a cell according to claim 19 or 20 under optimal conditions for expression of the specific binding molecule chains and b) isolating the specific binding molecule chains.

25. A method of treating cancer, comprising administering to a subject in need thereof a therapeutically effective amount of a specific binding molecule of any one of claims 1 to 13 or a specific binding molecule-anti-CD3 fusion molecule according to any one of claims 14-16.


SEQUENCE LISTING (45 sequences)

SEQ ID NO: 1 - protein, 10 aa, homo sapien
VVVGADGVGK

SEQ ID NO: 2 - protein, 203 aa, homo sapien
AQKVTQAQTEISVVEKEDVTLDCVYETRDTTYYLFWYKQPPSGELVFLIRRNSFDEQNEISGRYSWNFQKSTSSFNFTITASQVVDSAVYFCALSGPSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 3 - protein, 245 aa, homo sapien
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSYGPGQHNSPLHFGNGTRLTVTEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 4 - protein, 117 aa, artificial sequence
  Mutated TCR alpha variable domain (a96)
AQKVTQAQTEISVVEKEDVTLDCVYETRDTAYYLFWYKQPPSGELVFLIRQPWWGSSRGISGRYSWNFQKSTSSFNFTITASQVVDSAVYFCAMSVPDSRGHYQFTFGKGTKLSVIP

SEQ ID NO: 5 - protein, 117 aa, artificial sequence
  Mutated TCR alpha variable domain (a95)
AQKVTQAQTEISVVEKEDVTLDCVYETRDTAYYLFWYKQPPSGELVFLIRQPWWGSSRGISGRYSWNFQKSTSSFNFTITASQVVDSAVYFCAMSVPDMEGHYQFTFGKGTKLSVIP

SEQ ID NO: 6 - protein, 117 aa, artificial sequence
  Mutated TCR alpha variable domain (a41)
AQKVTQAQTEISVVEKEDVTLDCVYETRDTAYYLFWYKQPPSGELVFLIRQPWWGEQNEISGRYSWNFQKSTSSFNFTITASQVVDSAVYFCAMSVPSGDGSYQFTFGKGTKLSVIP

SEQ ID NO: 7 - protein, 115 aa, artificial sequence
  Mutated TCR beta variable domain (b35)
NAGVTQTPKFRVLKTGQSMTLLCAQDMNHEYMYWYRQDPGMGLRLIHYSGWGKDTAKGEVPDGYNVSRLKKQNFLLGLESAAPSQTSVYFCASKVGPGQHNSPLHFGNGTRLTVT

SEQ ID NO: 8 - protein, 115 aa, artificial sequence
  Mutated TCR beta variable domain (b9)
NAGVTQTPKFRVLKTGQSMTLLCAQDMNHEYMYWYRQDPGMGLRLIHYSGWGKDTAKGEVPDGYNVSRLKKQNFLLGLESAAPSQTSVYFCASSYGPGQHNSPLHFGNGTRLTVT

SEQ ID NO: 9 - protein, 203 aa, artificial sequence
  TCR-antiCD3 fusion (a96b35U0) Alpha chain (a96)
AQKVTQAQTEISVVEKEDVTLDCVYETRDTAYYLFWYKQPPSGELVFLIRQPWWGSSRGISGRYSWNFQKSTSSFNFTITASQVVDSAVYFCAMSVPDSRGHYQFTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 10 - protein, 503 aa, artificial sequence
  TCR-antiCD3 fusion (a96b35U0) Beta chain (b35U0)
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSNAGVTQTPKFRVLKTGQSMTLLCAQDMNHEYMYWYRQDPGMGLRLIHYSGWGKDTAKGEVPDGYNVSRLKKQNFLLGLESAAPSQTSVYFCASKVGPGQHNSPLHFGNGTRLTVTEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 11 - protein, 503 aa, artificial sequence
  TCR-antiCD3 fusion (a96b35U28) Beta chain (b35U28)
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSSGGGGSNAGVTQTPKFRVLKTGQSMTLLCAQDMNHEYMYWYRQDPGMGLRLIHYSGWGKDTAKGEVPDGYNVSRLKKQNFLLGLESAAPSQTSVYFCASKVGPGQHNSPLHFGNGTRLTVTEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 12 - protein, 203 aa, artificial sequence
  TCR-antiCD3 fusion (a95b9U0) Alpha chain (a95)
AQKVTQAQTEISVVEKEDVTLDCVYETRDTAYYLFWYKQPPSGELVFLIRQPWWGSSRGISGRYSWNFQKSTSSFNFTITASQVVDSAVYFCAMSVPDMEGHYQFTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 13 - protein, 503 aa, artificial sequence
  TCR-antiCD3 fusion (a95b9U0) Beta chain (b9U0)
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSGGGGSNAGVTQTPKFRVLKTGQSMTLLCAQDMNHEYMYWYRQDPGMGLRLIHYSGWGKDTAKGEVPDGYNVSRLKKQNFLLGLESAAPSQTSVYFCASSYGPGQHNSPLHFGNGTRLTVTEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 14 - protein, 503 aa, artificial sequence
  TCR-antiCD3 fusion (a95b9U28) Beta chain (b9U28)
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSSGGGGSNAGVTQTPKFRVLKTGQSMTLLCAQDMNHEYMYWYRQDPGMGLRLIHYSGWGKDTAKGEVPDGYNVSRLKKQNFLLGLESAAPSQTSVYFCASSYGPGQHNSPLHFGNGTRLTVTEDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 15 - protein, 203 aa, artificial sequence
  TCR-antiCD3 fusion (a41b9U0) Alpha chain (a41)
AQKVTQAQTEISVVEKEDVTLDCVYETRDTAYYLFWYKQPPSGELVFLIRQPWWGEQNEISGRYSWNFQKSTSSFNFTITASQVVDSAVYFCAMSVPSGDGSYQFTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 16 - protein, 503 aa, artificial sequence; identical to SEQ ID NO: 13
  TCR-antiCD3 fusion (a41b9U0) Beta chain (b9U0)

SEQ ID NO: 17 - protein, 503 aa, artificial sequence; identical to SEQ ID NO: 14
  TCR-antiCD3 fusion (a41b9U28) Beta chain (b9U28)

SEQ ID NO: 18 - protein, 5 aa, artificial sequence
  Linker
GGGGS

SEQ ID NO: 19 - protein, 5 aa, artificial sequence
  Linker
GGGSG

SEQ ID NO: 20 - protein, 5 aa, artificial sequence
  Linker
GGSGG

SEQ ID NO: 21 - protein, 5 aa, artificial sequence
  Linker
GSGGG

SEQ ID NO: 22 - protein, 6 aa, artificial sequence
  Linker
GSGGGP

SEQ ID NO: 23 - protein, 5 aa, artificial sequence
  Linker
GGEPS

SEQ ID NO: 24 - protein, 7 aa, artificial sequence
  Linker
GGEGGGP

SEQ ID NO: 25 - protein, 12 aa, artificial sequence
  Linker
GGEGGGSEGGGS

SEQ ID NO: 26 - protein, 8 aa, artificial sequence
  Linker
GGGSGGGG

SEQ ID NO: 27 - protein, 4 aa, artificial sequence
  Linker
GGGS

SEQ ID NO: 28 - protein, 5 aa, artificial sequence
  Linker
GGGGS

SEQ ID NO: 29 - protein, 5 aa, artificial sequence
  Linker
TVLRT

SEQ ID NO: 30 - protein, 6 aa, artificial sequence
  Linker
TVSSAS

SEQ ID NO: 31 - protein, 7 aa, artificial sequence
  Linker
TVLSSAS

SEQ ID NO: 32 - protein, 7 aa, artificial sequence
  TCR alpha chain CDR1
TRDTTYY

SEQ ID NO: 33 - protein, 9 aa, artificial sequence
  TCR alpha chain CDR2
RNSFDEQNE

SEQ ID NO: 34 - protein, 16 aa, artificial sequence
  TCR alpha chain CDR3
CALSGPSGAGSYQLTF

SEQ ID NO: 35 - protein, 5 aa, artificial sequence
  TCR beta chain CDR1
MNHEY

SEQ ID NO: 36 - protein, 6 aa, artificial sequence
  TCR beta chain CDR2
SVGEGT

SEQ ID NO: 37 - protein, 16 aa, artificial sequence
  TCR beta chain CDR3
CASSYGPGQHNSPLHF

SEQ ID NO: 38 - protein, 7 aa, artificial sequence
  TCR alpha chain CDR1
TRDTAYY

SEQ ID NO: 39 - protein, 9 aa, artificial sequence
  TCR alpha chain CDR2
QPWWGSSRG

SEQ ID NO: 40 - protein, 9 aa, artificial sequence
  TCR alpha chain CDR2
QPWWGEQNE

SEQ ID NO: 41 - protein, 16 aa, artificial sequence
  TCR alpha chain CDR3
CAMSVPDSRGHYQFTF

SEQ ID NO: 42 - protein, 16 aa, artificial sequence
  TCR alpha chain CDR3
CAMSVPDMEGHYQFTF

SEQ ID NO: 43 - protein, 16 aa, artificial sequence
  TCR alpha chain CDR3
CAMSVPSGDGSYQFTF

SEQ ID NO: 44 - protein, 6 aa, artificial sequence
  TCR beta chain CDR2
SGWGKD

SEQ ID NO: 45 - protein, 16 aa, artificial sequence
  TCR beta chain CDR3
CASKVGPGQHNSPLHF